(19) United States
(12) Patent Application Publication
(10) Pub. No.: US 2023/0157979 A1
(43) Pub. Date: May 25, 2023

(54) TOPICAL DICLOFENAC COMPOSITIONS AND METHODS

(71) Applicant: Ferring B.V., Hoofddorp (NL)

(72) Inventors: Dario N.R. CARRARA, Saint-Prex (CH); Arnaud Grenier, Saint-Prex (CH)

(73) Assignee: Ferring B.V., Hoofddorp (NL)

(21) Appl. No.: 17/801,703

(22) PCT Filed: Feb. 25, 2021
(86) PCT No.: PCT/NL2021/050125

§ 371 Date: Aug. 23, 2022

Related U.S. Application Data
(60) Provisional application No. 62/982,589, filed on Feb. 27, 2020.

(51) Int. Cl. A61K 31/196; A61K 9/00; A61K 47/10; A61K 9/06; A61P 19/02

(52) U.S. Cl. CPC A61K 31/196; A61K 9/0014; A61K 47/10; A61K 9/06; A61P 19/02

(57) ABSTRACT

Described herein are pharmaceutical compositions comprising diclofenac and therapeutic methods for using them effective with once daily administration.

### CROSS-REFERENCE TO RELATED APPLICATIONS

This application claims priority under 35 U.S.C. § 119(e) to U.S. provisional application 62/982,589, filed Feb. 27, 2020, the entire contents of which are incorporated herein by reference in their entirety.

### FIELD

Described are topical diclofenac compositions and therapeutic methods using them.

### BACKGROUND

Diclofenac (2-(2,6-dichloranilino) phenylacetic acid) is a non-steroidal anti-inflammatory drug (NSAID) used to reduce inflammation and, as an analgesic, to reduce pain. It is available in sodium, potassium, epolamine or diethylamine salt form in numerous dosage forms (oral tablet, oral syrup, topical gel, cataplasm, ophthalmic drop, suppository, etc.).

An example of a well-known topical/transdermal diclofenac formulation is Voltaren® Gel 1% which comprises 1% diclofenac sodium. Voltaren® Gel 1% is indicated in the U.S. for the relief of the pain due to osteoarthritis of joints amenable to topical treatment, such as the knees and the hands. Up to 4 g of Voltaren® Gel 1% can be applied to lower extremities (including the knees, the ankles, and the feet) 4 times daily so that up to not more than 16 g daily of Voltaren® Gel 1% is applied to any single joint of the lower extremities. Up to 2 g of Voltaren® Gel 1% can be applied to the upper extremities (which include the elbows, the wrists and the hands) 4 times daily so that up to not more than 8 g daily of Voltaren® Gel 1% is applied to any single joint of the upper extremities. Overall, the total dose of Voltaren® Gel 1% should not exceed 32 g per day over all affected joints. Neither the total amount (up to 32 g per day) nor the frequency of application (4 times a day) are satisfactory from a patient perspective.

U.S. Pat. No. 7,335,379 discloses formulations for transdermal or transmucosal administration of active agents, such as diclofenac, containing an alkanol, a polyalcohol, a monoalkyl ether of diethylene glycol and a fatty alcohol with a fatty alcohol content of up to 2%.

U.S. Pat. Nos. 9,999,590 and 10,117,829 disclose transdermal or transmucosal formulations of diclofenac comprising, e.g., at least 3% weight/weight diclofenac, a C2 to C4 alkanol, a polyalcohol, a monoalkyl ether of diethylene glycol, and at least 3% weight/weight of a fatty alcohol.

Osteoarthritis is the most common form of arthritis and is one of the leading causes of disability in the world, with more than 10% of the population aged 50 and above having symptoms of the disease. The hallmark of the disease is joint pain, reduced physical function and progressive degeneration of articular cartilage. Available drugs to reduce the symptomatic burden of osteoarthritis include oral and topical NSAIDs, paracetamol, opioids and intra-articular corticosteroid injections. NSAIDs, including diclofenac, inhibit the COX enzyme, which reduces pain and inflammation through the inhibition of prostaglandins. However, currently approved topical formulations of NSAIDs such as Voltaren® Gel 1% require several daily applications of considerable amounts of gel to achieve a modest efficacy.

The diclofenac molecule possesses excellent physicochemical properties for skin and deep tissue penetration. While the systemic absorption of topically applied diclofenac is low, the concentration of diclofenac has been reported to be higher in the local muscle tissue after topical administration compared to systemically administered diclofenac. Clinical trials of currently approved diclofenac sodium 1% gel formulations found that four daily applications were required to demonstrate a statistically significant efficacy effect compared to placebo.

Thus, there remains a need for topical diclofenac compositions and therapeutic methods using them, particularly a need for topical diclofenac compositions and therapeutic methods that are effective with once daily administration.

### SUMMARY

Provided in one aspect is a method for treating signs and symptoms of osteoarthritis of a joint amenable to topical treatment, comprising topically applying once daily to an affected area of a subject in need thereof a therapeutically effective amount of a topical diclofenac composition, wherein the composition comprises (i) at least 2.7% weight/weight of diclofenac; (ii) a C2 to C4 alkanol; (iii) a polyalcohol; (iv) a monoalkyl ether of diethylene glycol; and (v) from about 3% to about 5% weight/weight of a fatty alcohol, all based on the total weight of the topical diclofenac composition, wherein the once daily application provides a daily dose of diclofenac per joint of from about 30 mg to about 100 mg, including a daily dose of diclofenac per joint of 90-110 mg, based on diclofenac sodium. In some embodiments, the topical diclofenac composition further comprises at least one of a gelling agent, an anti-oxidant, a solvent, and any combinations thereof.

In some embodiments, the method is effective for relief of pain of osteoarthritis. In some embodiments, the method is effective for relief of pain of osteoarthritis as determined by Western Ontario and McMaster Osteoarthritis Index (WOMAC) assessment before and after treatment. In some embodiments, the method is effective for relief of pain of osteoarthritis as determined by a decrease in the subject's WOMAC pain sub-score after treatment as compared to before treatment. In some embodiments, the method is effective for relief of pain of osteoarthritis as determined by a decrease in one or more of the subject's WOMAC physical function sub-score and WOMAC stiffness sub-score after treatment as compared to before treatment. In some embodiments, the method is effective for relief of pain of osteoarthritis as determined by a decrease in the subject's WOMAC weight-bearing pain sub-score after treatment as compared to before treatment. In some embodiments, the method is effective for relief of pain of osteoarthritis as determined by a decrease in the subject's WOMAC non-weight-bearing pain sub-score after treatment as compared to before treatment. In some embodiments, the method is effective for relief of pain of osteoarthritis as determined by a decrease in the subject's WOMAC total score after treatment as compared to before treatment. In some embodiments, before treatment the subject has a WOMAC pain sub-score of 40 or greater when normalized to a scale of 0 to 100. In some embodiments, before treatment the subject has a WOMAC pain sub-score of 40 or greater and 90 or less when normalized to a scale of 0 to 100. In some embodiments, the method is at least as effective for relief of pain of osteoarthritis as compared to twice daily administration of the same amount of the same topical diclofenac composition, as determined by WOMAC pain sub-score assessment before and after treatment. In some embodiments, WOMAC assessment after treatment is conducted after treatment once daily for a period of time selected from 1-4 weeks, 4-8 weeks, 8-12, weeks, or longer.

In some embodiments, the topical diclofenac composition comprises the diclofenac in an amount of about 3% weight/weight; the C2 to C4 alkanol in an amount of about 5-60% weight/weight; the polyalcohol in an amount of about 1-30% weight/weight; the monoalkyl ether of diethylene glycol in an amount of about 0.2-25% weight/weight; a gelling agent in an amount of about 0.05-5% weight/weight, and an antioxidant in an amount of about 0.025-2.0% weight/weight, all based on the total weight of the topical diclofenac composition. In some embodiments, the topical diclofenac composition comprises a C2 to C4 alkanol selected from ethanol, isopropanol, n-propanol, butan-1-ol, and butan-2-ol; a polyalcohol selected from glycol, propylene glycol, butylene glycol, and hexylene glycol; a monoalkyl ether of diethylene glycol selected from diethylene glycol monoethyl ether, diethylene glycol monomethyl ether, and combinations thereof; and a fatty alcohol selected from myristyl alcohol, lauryl alcohol, oleyl alcohol, cetyl alcohol, and stearyl alcohol.

In some embodiments, the topical diclofenac composition comprises diclofenac sodium. In some embodiments, the topical diclofenac composition comprises diclofenac sodium in an amount from 2.7-3.3% weight/weight; ethanol in an amount from 40.5-49.5% weight/weight; propylene glycol in an amount from 18-22% weight/weight; diethylene glycol monoethyl ether in an amount from 4.5-5.5% weight/weight; and myristyl alcohol in an amount from 2.7-3.3% weight/weight. In some embodiments, the topical diclofenac composition comprises diclofenac sodium in an amount of about 3.0% weight/weight; ethanol in an amount from 40.5-49.5% weight/weight; propylene glycol in an amount from 18-22% weight/weight; diethylene glycol monoethyl ether in an amount from 4.5-5.5% weight/weight; and myristyl alcohol in an amount from 2.7-3.3% weight/weight. In some embodiments, the topical diclofenac composition comprises diclofenac sodium in an amount from 2.7-3.3% weight/weight; ethanol in an amount from 40.5-49.5% weight/weight; propylene glycol in an amount from 18-22% weight/weight; diethylene glycol monoethyl ether in an amount from 4.5-5.5% weight/weight; myristyl alcohol in an amount from 2.7-3.3% weight/weight; hydroxypropyl cellulose in an amount from 1.25-1.75% weight/weight; and water. In some embodiments, the topical diclofenac composition comprises diclofenac sodium in an amount of 3.06% weight/weight; ethanol in an amount of 46.0% weight/weight; propylene glycol in an amount of 20% weight/weight; diethylene glycol monoethyl ether in an of 5.0% weight/weight; myristyl alcohol in an amount of 3.0% weight/weight; hydroxypropyl cellulose in an amount of 1.5% weight/weight; and water.

In some embodiments, the therapeutically effective amount of the topical diclofenac composition applied once daily per joint is from about 0.75 to about 2.50 grams, or from about 0.75 to about 3.50 grams. In some embodiments, the joint is selected from a knee, elbow, or joint of a foot, ankle, hand, wrist, hip, shoulder, or spine. In some embodiments, the joint is a knee, hip, shoulder or joint of the spine, and the once daily application provides a daily dose of diclofenac per joint of from about 60 mg to about 100 mg, including a daily dose of diclofenac per joint of 90-110 mg, based on diclofenac sodium. In some embodiments, the once daily application provides a daily dose of diclofenac of about 70 mg, based on diclofenac sodium, per knee, or per hip, shoulder or joint of the spine. In some embodiments, the therapeutically effective amount of the topical diclofenac composition applied once daily to a knee, or to a hip, shoulder or joint of the spine, is from about 1.5 to about 2.5 grams, or from about 0.75 to about 3.50 grams. In some embodiments, the joint is an elbow, or joint of a foot, ankle, hand, or wrist, and the once daily application provides a daily dose of diclofenac per joint of from about 30 mg to about 70 mg, based on diclofenac sodium. In some embodiments, the once daily application to an elbow, or joint of a foot, ankle, hand, or wrist, provides a daily dose of diclofenac of about 35 mg per joint, based on diclofenac sodium. In some embodiments, the therapeutically effective amount of the topical diclofenac composition applied once daily to an elbow, or joint of a foot, ankle, hand, or wrist is from about 0.75 to about 1.50 grams.

In some embodiments, the topical diclofenac composition is in a form selected from a gel, lotion, cream, spray, aerosol, ointment, emulsion, suspension, liposomal system, lacquer, patch, bandage, and occlusive dressing. In some embodiments, the topical diclofenac composition is in a form selected from a gel, lotion, cream, spray, aerosol, ointment, emulsion, suspension, liposomal system, and lacquer. In some embodiments, the topical diclofenac composition is in the form of a gel.

In some embodiments, the topical diclofenac gel composition in a form selected from a gel, lotion, cream, spray, aerosol, ointment, emulsion, suspension, liposomal system, or lacquer is applied from a metered dose dispenser. In some embodiments, the metered dose dispenser comprises a metered dose pump. In some embodiments, the therapeutically effective amount of the topical diclofenac composition is provided by one or two or three or more actuations of the metered dose pump.

Provided in another aspect is a kit for once daily treatment of signs and symptoms of osteoarthritis of a joint amenable to topical treatment, comprising at least one container containing a topical diclofenac composition as described herein, such as a topical diclofenac composition that comprises diclofenac sodium in an amount of 3.06% weight/weight; ethanol in an amount of 46.0% weight/weight; propylene glycol in an amount of 20% weight/weight; diethylene glycol monoethyl ether in an of 5.0% weight/weight; myristyl alcohol in an amount of 3.0% weight/weight; and hydroxypropyl cellulose in an amount of 1.5% weight/weight; and water. In some embodiments, the kit further comprises instructions for topically applying once daily to an affected area of a subject in need thereof a therapeutically effective amount of the composition that provides a daily dose of the diclofenac sodium of from about 30 mg to about 100 mg per joint, including a daily dose of 90-110 mg per joint. In some embodiments, the container is a metered dose dispenser. In some embodiments, the metered dose dispenser comprises a metered dose pump. In some embodiments, the therapeutically effective amount of the topical diclofenac composition is provided by one or two or more actuations of the metered dose pump.

### BRIEF DESCRIPTION OF THE DRAWINGS

FIG. 1 shows a flow chart of the preparation of a diclofenac gel composition as described herein, suitable for use in the methods described herein.

FIG. 2 shows the trial design of the clinical trial study described in Example 2.

FIG. 3 is the Western Ontario and McMaster Osteoarthritis (WOMAC) questionnaire used in the clinical trial study described in Example 2.

FIG. 4 shows the change from baseline in Western Ontario and McMaster Osteoarthritis (WOMAC) pain sub-score results from Example 2.

FIG. 5 shows the change from baseline in WOMAC pain sub-score results from the post-hoc analysis of the sub-population of subjects who had a WOMAC score ≥40 out of 100 at screening and at baseline from Example 2.

FIG. 6 shows the change from baseline in WOMAC pain sub-score results during the first week of treatment (day 0 to day 7) from Example 2.

FIG. 7 shows the change from baseline in WOMAC function sub-score score results from Example 2.

### DETAILED DESCRIPTION

Described are topical diclofenac compositions and therapeutic methods using them that are effective with once daily administration.

### Definitions

Technical and scientific terms used herein have the meanings commonly understood by one of ordinary skill in the art, unless otherwise defined.

As used herein, the singular forms “a,” “an,” and “the” designate both the singular and the plural, unless expressly stated to designate the singular only.

As used herein, the term “about” when qualifying a number or range means that the number or range is not limited to the exact number or range set forth, but encompass values around the stated number or range as will be understood by persons of ordinary skill in the art depending on the context in which the number or range is used. When not otherwise apparent from the context or convention in the art, “about” means up to plus or minus 10% of the particular term (±10%). Thus, for example, as used herein, disclosure of a daily dose of diclofenac per joint of from about 30 mg to about 100 mg includes the disclosure of a daily dose of diclofenac per joint of from about 30 mg to 110 mg, and the disclosure of a daily dose of diclofenac per joint of about 100 mg includes the disclosure of a daily dose of diclofenac per joint of 90-110 mg.

The terms “administer,” “administration,” or “administering” as used herein refer to providing, giving, dosing and/or prescribing, such as by either a health professional or his or her authorized agent or under his or her direction, and putting into, taking or consuming, such as by a health professional or the subject or patient.

The terms “subject” and “patient” as used herein refer to any mammal, including, but not limited to, humans, pets and laboratory animals (e.g., dogs, cats, rodents, rabbits, guinea pigs, primates, etc.), and farm animals and livestock (e.g., horses, camels, donkeys, cattle, sheep, pigs, goats, etc.).

As used herein, the phrase “therapeutically effective amount” refers to a dose that provides or has been determined to provide the specific pharmacological effect for which the drug is administered in a subject in need of such treatment, such as relief of pain. However, a “therapeutically effective amount” may not always be effective in treating the condition in a given subject, even though such dose is deemed to be a therapeutically effective amount by those of skill in the art. Exemplary doses and therapeutically effective amounts are provided below with reference to adult human subjects. Those skilled in the art can adjust such amounts in accordance with standard practices as needed to treat a specific subject and/or condition.

### Topical Diclofenac Compositions

The diclofenac compositions described herein may be applied topically for local or systemic effect. In either case, the compositions are effective to deliver diclofenac through the skin to which it is applied into target tissue (for local effect) and/or the bloodstream (for systemic effect). In specific embodiments, the compositions are used for treating signs and symptoms of osteoarthritis of a joint amenable to topical treatment, and are applied topically to an affected area of the subject (e.g., skin surface over the joint), for delivery into target tissue (e.g., synovial fluid of the joint), such as being topically applied to the knee for treatment of osteoarthritis knee pain.

In some embodiments, a topical diclofenac composition as described herein comprises (i) at least 2.7% weight/weight of diclofenac; (ii) a C2 to C4 alkanol; (iii) a polyalcohol; (iv) a monoalkyl ether of diethylene glycol; and (v) from about 3% to about 5% weight/weight of a fatty alcohol, all based on the total weight of the topical diclofenac composition. The compositions may further comprise one or more of a gelling agent, an anti-oxidant, and/or other components discussed below.

### Diclofenac

As noted above, the compositions described herein include diclofenac. The chemical name for diclofenac is 2-(2,6-dichloranilino) phenylacetic acid (CAS Registry Number 15307-86-5). Pharmaceutically acceptable salts of diclofenac include the sodium salt, potassium salt, epolamine salt, diethylamine salt, and any other pharmaceutically acceptable salt thereof. In some embodiments, compositions as described herein include diclofenac sodium. The chemical formula for diclofenac sodium is C₁₄H₁₀C₁₂NNaO₂. Unless stated otherwise, “diclofenac” as used herein refers to 2-(2,6-dichloranilino) phenylacetic acid and any pharmaceutically acceptable salts thereof.

As noted above, the compositions as described herein comprise at least 2.7% weight/weight diclofenac. In specific embodiments, the compositions may comprise from 2.7% to 3.3% weight/weight diclofenac. As non-limiting examples, the composition may comprise diclofenac in an amount of 2.7%, 2.8%, 2.9%, 3.0%, 3.1%, 3.2%, or 3.3%, weight/weight. In some embodiments, the composition comprises about 3% weight/weight of diclofenac. In some embodiments, the composition comprises 3.06% weight/weight of diclofenac. In specific embodiments comprising diclofenac sodium, the compositions may comprise at least 2.7% weight/weight diclofenac sodium. For example, the compositions may comprise from 2.7% to 3.3% weight/weight diclofenac sodium. As non-limiting examples, the compositions may comprise diclofenac sodium in an amount of 2.7%, 2.8%, 2.9%, 3.0%, 3.1%, 3.2%, or 3.3% weight/weight. In some embodiments, the compositions may comprise about 3% weight/weight of diclofenac sodium. In some embodiments, the compositions comprise 3.06% weight/weight of diclofenac sodium.

### C2-C4 Alkanol

As noted above, the compositions described herein comprise a C2 to C4 alkanol. The term “C2 to C4 alkanol” as used herein refers to one or more C2 to C4 alkanes substituted with a hydroxy group (—OH). Non-limiting examples of C2 to C4 alkanols include ethanol, isopropanol, n-propanol, butan-1-ol and butan-2-ol. In some embodiments, the C2 to C4 alkanol is ethanol.

The composition described herein may comprise a C2 to C4 alkanol in an amount from about 5% to about 60% weight/weight (e.g., about 5, 10, 15, 20, 25, 30, 35, 40, 45, 50, 55, or 60% weight/weight). For example, the composition may include a C2 to C4 alkanol in an amount from about 40% to about 50% weight/weight, including from 40.5% to 49.5% weight/weight, such as 46.0% weight/weight. In specific embodiments comprising ethanol, the composition may comprise ethanol in an amount from about 5% to about 60% weight/weight (e.g., about 5, 10, 15, 20, 25, 30, 35, 40, 45, 50, 55, or 60% weight/weight). For example, the composition may include ethanol in an amount from about 40% to about 50% weight/weight, including from 40.5% to 49.5% weight/weight, such as 46.0% weight/weight. In embodiments comprising ethanol, the composition may be formulated with absolute ethanol or, for example, an amount of 96% v/v ethanol to provide an equivalent amount of ethanol.

In some embodiments, the C2 to C4 alkanol (such as for example ethanol) serves as the primary solvent for the diclofenac in the composition. In such embodiments, the quantity of the C2 to C4 alkanol is sufficient to at least fully solubilize the diclofenac. Additionally or alternatively, in some embodiments, the C2 to C4 alkanol used, such as ethanol, also serves as an efficient skin permeation enhancer for diclofenac. In some embodiments, the composition may further comprise an additional solvent, as discussed below. In any embodiments described herein, the composition may comprise a C2 to C4 alkanol in a hydroalcoholic mixture with water.

### Polyalcohol

As noted above, the compositions described herein comprise a polyalcohol. The term “polyalcohol” as used herein refers to one or more C2 to C6 alkanes or C2 to C6 alkenes, substituted with two or more hydroxy groups. Non-limiting examples of polyalcohols include, but are not limited to, ethylene glycol, propylene glycol, butylene glycol, and hexylene glycol. In some embodiments, the polyalcohol is propylene glycol.

The composition described herein may comprise a polyalcohol in an amount from about 1% to about 30% weight/weight (e.g., about 1, 2, 3, 4, 5, 10, 15, 20, 25, or 30% weight/weight). For example, the composition may include a polyalcohol in an amount from 18% to 22% weight/weight, such as 20% weight/weight. In specific embodiments comprising propylene glycol, the composition may comprise propylene glycol in an amount from about 1% to about 30% weight/weight (e.g., about 1, 2, 3, 4, 5, 10, 15, 20, 25, or 30% weight/weight). For example, the composition may include propylene glycol in an amount from 18% to 22% weight/weight, such as 20% weight/weight.

### Monoalkyl Ether of Diethylene Glycol

As noted above, the compositions described herein comprise a monoalkyl ether of diethylene glycol. The term “monoalkyl ether of diethylene glycol” as used herein refers to one or more diethylene glycol moieties substituted with a C1 to C6 alkyl ether. Non-limiting examples of monoalkyl ethers diethylene glycol include, but are not limited to, one or both of diethylene glycol monoethyl ether (DOME) and diethylene glycol monomethyl ether (DGMM). In some embodiments, the monoalkyl ether of diethylene glycol is diethylene glycol monoethyl ether.

The composition may comprise a monoalkyl ether of diethylene glycol in an amount from about 0.2% to about 25% weight/weight (e.g., about 0.2, 0.3, 0.4, 0.5, 0.6, 0.7, 0.8, 0.9, 1.0, 2.0, 3.0, 4.0, 5.0, 6.0, 7.0, 8.0, 9.0, 10, 15, 20, or 25% weight/weight). For example, the composition may include a monoalkyl ether of diethylene glycol in an amount from 4.5% to 5.5% weight/weight, such as 5% weight/weight. In specific embodiments comprising diethylene glycol monoethyl ether, the composition may comprise diethylene glycol monoethyl ether in an amount from about 0.2% to about 25% weight/weight (e.g., about 0.2, 0.3, 0.4, 0.5, 0.6, 0.7, 0.8, 0.9, 1.0, 2.0, 3.0, 4.0, 5.0, 6.0, 7.0, 8.0, 9.0, 10, 15, 20, or 25% weight/weight). For example, the composition may include diethylene glycol monoethyl ether in an amount from 4.5% to 5.5% weight/weight, such as 5% weight/weight.

### Fatty Alcohol

As noted above, the compositions described herein comprise a fatty alcohol. The term “fatty alcohol” as used herein refers to a fatty alcohol having a branched or linear carbon body having 12 or more carbon atoms. Non-limiting examples of fatty alcohols include, but are not limited to, myristyl alcohol, lauryl alcohol, oleyl alcohol, cetyl alcohol, and stearyl alcohol. In some embodiments, the fatty alcohol is myristyl alcohol.

The composition may comprise a fatty alcohol in an amount from about 3% to about 5% weight/weight (e.g., about 3.0, 3.5, 4.0, 4.5, or 5.0% weight/weight). For example, the composition may comprise a fatty alcohol in an amount of 2.7% to 3.3% weight/weight, such as 3.0% weight/weight. In specific embodiments comprising myristyl alcohol, the composition may comprise myristyl alcohol in an amount from about 3% to about 5% weight/weight (e.g., about 3.0, 3.5, 4.0, 4.5, or 5.0% weight/weight). For example, the composition may comprise myristyl alcohol in an amount of 2.7% to 3.3% weight/weight, such as 3.0% weight/weight. As used herein, and in accordance with the definitions in the United States Pharmacopeia (USP) and National Formulary (NF), “myristyl alcohol” refers to a product that contain not less than 90.0% myristyl alcohol (CH₃(CH₂)₁₃O), the remainder consisting chiefly of related alcohols.

In any of the embodiments disclosed herein, the composition may further comprise a fatty ester having a branched or linear acid moiety having 12 or more carbon atoms or having a branched or linear alcohol moiety having 12 or more carbon atoms.

While not wanting to be bound by theory, it is believed that, in any of the embodiments disclosed herein, the combination of C2 to C4 alkanol, polyalcohol, monoalkyl ether of diethylene glycol, and fatty alcohol forms a “permeation enhancing system,” which qualitatively and/or quantitatively enhances the absorption and/or permeation of diclofenac through the skin to which it is applied, as compared to a topical diclofenac composition formulated without said permeation enhancing system.

### Gelling Agent

The compositions described herein may optionally comprise a gelling agent. The term “gelling agent” as used herein refers to any agent capable of transforming the composition into a gel. Examples of gelling agents include, but are not limited to, one or more selected from carbomers (also known as carboxyethylene or polyacrylic acid) such as CARBOPOL® carbomers, such as CARBOPOL® 980NF or 940 NF, CARBOPOL® 981 or 941 NF, CARBOPOL® 1382 or 1342 NF, CARBOPOL® 5984 or 934 NF, CARBOPOL® ETD 2020, CARBOPOL® 934P NF, CARBOPOL® 971P NF, CARBOPOL® 974P NF, CARBOPOL® 71G NF, CARBOPOL® Ultrez 10 NF, PEMULEN® TR-1 NF or TR-2 NF, and NOVEON® AA-1 USP, cellulose derivatives such as ethylcellulose (EC), hydroxypropylmethylcellulose (HPMC), ethylhydroxyethylcellulose (EHEC), carboxymethylcellulose (CMC), hydroxypropylcellulose (HPC) (e.g., KLUCEL™ grades), hydroxyethylcellulose (HEC) (e.g., NATROSOL® grades), hydroxypropyl methylcellulose phthalate (e.g., HPMC-P 55), and methylcellulose (e.g., METHOCEL® grades), natural gums such as arabic, xanthan, guar gums, and alginates, polyvinylpyrrolidone (PVP) and PVP derivatives (e.g., KOLLIDON® grades), polyoxyethylene-polyoxypropylene copolymers (e.g., LUTROL® F grades 68 and 127), chitosan, polyvinyl alcohols, pectins, and smectite clays (e.g., VEEGUM® grades). Additionally or alternatively, tertiary amines, such as one or more of triethanolamine, trolamine, tromethamine, aminomethyl propanol, diisopropanolamine, and diethylamine, may be used to thicken and/or neutralize the system.

In some embodiments, the gelling agent is a carbomer. The term “carbomer” refers to a class of homopolymers of acrylic acid with a high molecular weight optionally cross-linked with any of several polyalcohol allyl ethers, such as an allyl ether of pentaerythritol, allyl ether of sucrose, or allyl ether of propylene. Non-limiting examples of carbomers are carbomer 940, carbomer 971P, carbomer 973, carbomer 974P, carbomer 980NF, and carbomer C981 NF (wherein the digit indicates the average molecular weight of the polymer chains).

In some embodiments, a gelling agent is present and is hydroxypropyl cellulose. In some embodiments, a gelling agent is present and is a carbomer.

The identity and amount of gelling agent may be selected to provide a composition having a viscosity particularly suitable for a topical gel composition, such as a viscosity of from about 8000 cPs (mPa·s) to about 14000 cPs (mPa·s), including about 10000 cPs (mPa·s). For example, from 1.25% to 1.75% weight/weight of KLUCEL™ hydroxypropylcellulose HF grade can be used to provide a composition as described herein with a viscosity of about 8000 cPs (mPa·s) to about 14000 cPs (mPa·s). Those of ordinary skill in the art can select and use suitable amounts of other hydroxypropylcelluloses to achieve a composition having a suitable target viscosity using knowledge in the art and/or routine screening methods. Similarly, those of ordinary skill in the art can select and use suitable amounts of other gelling agents, including one or more of those listed above, to achieve a composition having a suitable target viscosity using knowledge in the art and/or routine screening methods.

Thus, the composition optionally may comprise a gelling agent in an amount from about 0.05% to about 5% weight/weight (e.g., about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, or 5% weight/weight). For example, the composition may comprise a gelling agent in an amount of 1.25% to 1.75% weight/weight, such as 1.5% weight/weight. In specific embodiments comprising hydroxypropyl cellulose, the composition may comprise hydroxypropyl cellulose (such as, for example, KLUCEL™ hydroxypropylcellulose HF grade) in an amount from about 0.05% to about 5% weight/weight (e.g., about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, or 5% weight/weight). For example, the composition may comprise hydroxypropyl cellulose in an amount of 1.25% to 1.75% weight/weight, such as 1.5% weight/weight. In specific embodiments comprising a carbomer, the composition may comprise a carbomer (such as, for example CARBOPOL® ETD 2020 polymer) in an amount from about 0.05% to about 5% weight/weight (e.g., about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, or 5% weight/weight). For example, the composition may comprise a carbomer in an amount from 0.5% to 2.0% weight/weight, such as 1% weight/weight.

### Anti-Oxidant

The compositions described herein may optionally comprise an anti-oxidant or stabilizer. The terms “anti-oxidant” or “stabilizer” are used herein interchangeably to refer to any substance capable of preventing oxidation or reactions promoted by oxygen, peroxides, or free radicals in a formulation. Examples of suitable anti-oxidants include one or more selected from tocopherol and derivatives thereof, ascorbic acid and derivatives thereof, butylated hydroxyanisole, butylated hydroxytoluene, fumaric acid, malic acid, citric acid, propyl gallate, sodium metabisulfite, sodium bisulfite, sodium sulfite, edetate disodium, edetate trisodium, edetate tetrasodium, and derivatives thereof. In some embodiments, an anti-oxidant is present and is or comprises sodium metabisulfite. In some embodiments, an anti-oxidant is present and is or comprises propyl gallate. In some embodiments, an anti-oxidant is present and is or comprises edetate tetrasodium.

Thus, the composition may optionally comprise an anti-oxidant, typically in an amount of about 0.025% to about 2% weight/weight (e.g., about 0.025, 0.03, 0.035, 0.04, 0.045, 0.05, 0.055, 0.06, 0.065, 0.07, 0.075, 0.08, 0.085, 0.09, 0.095, 0.1%, 0.15%, or 0.2% weight/weight). For example, the composition may comprise an anti-oxidant in an amount of 0.025% to 0.10% weight/weight, or in an amount of up to 0.05% weight/weight. In specific embodiments, the composition comprises sodium metabisulfite in an amount from about 0.025% to about 0.10% weight/weight. Additionally or alternatively, in specific embodiments, the composition comprises propyl gallate in an amount of up to about 0.05% weight/weight. Additionally or alternatively, in specific embodiments, the composition comprises edetate tetrasodium in an amount of up to about 0.05% weight/weight. Additionally or alternatively, in specific embodiments, the composition comprises sodium metabisulfite in an amount from about 0.025% to about 0.10% weight/weight and propyl gallate in an amount of up to about 0.05% weight/weight. Additionally or alternatively, in specific embodiments, the composition comprises sodium metabisulfite in an amount from about 0.025% to 0.10% weight/weight and edetate tetrasodium in an amount of up to about 0.05% weight/weight.

### Additional Solvent

As noted above, in some embodiments, the C2 to C4 alkanol (such as for example ethanol) serves as the primary solvent for the diclofenac in the composition. In some embodiments, one or more of the other components listed above also acts as a solvent or cosolvent for the diclofenac. In some embodiments, the composition may further comprise an additional solvent. A “solvent” as used herein may encompass any type of solvent suitable for use in a topical formulation as described herein, including water.

### Additional Excipients

In some embodiments, the compositions disclosed herein further comprise one or more additional components or excipients suitable for use in a topical pharmaceutical composition, such as one or more of a buffering agent, moisturizing agent, humectant, surfactant, neutralizing agent, chelating agent, emollient, fragrance, or the like.

### Exemplary Compositions

A topical diclofenac composition as disclosed herein may include any amount of diclofenac described above, any amount of any C2 to C4 alkanol described above, any amount of any polyalcohol described above, any amount of any monoalkyl ether of diethylene glycol described above, and any amount of any fatty alcohol as described above, and, optionally, any amount of gelling agents(s), anti-oxidants(s), additional solvent(s), and/or other excipients described above, in any combination thereof. The following are disclosed as specific illustrative embodiments.

In some embodiments, the topical diclofenac composition comprises (i) at least 2.7% weight/weight of diclofenac; (ii) a C2 to C4 alkanol; (iii) a polyalcohol; (iv) a monoalkyl ether of diethylene glycol; and (v) from about 3% to about 5% weight/weight of a fatty alcohol, all based on the total weight of the topical diclofenac composition. In some embodiments, the C2 to C4 alkanol is selected from ethanol, isopropanol, n-propanol, butan-1-ol, and butan-2-ol; the polyalcohol is selected from glycol, propylene glycol, butylene glycol, and hexylene glycol; the monoalkyl ether of diethylene glycol is selected from diethylene glycol monoethyl ether, diethylene glycol monomethyl ether, and combinations thereof; and the fatty alcohol is selected from myristyl alcohol, lauryl alcohol, oleyl alcohol, cetyl alcohol, and stearyl alcohol.

In some embodiments, the topical diclofenac composition comprises diclofenac in an amount of about 3% weight/weight; the C2 to C4 alkanol in an amount of about 5-60% weight/weight; the polyalcohol in an amount of about 1-30% weight/weight; the monoalkyl ether of diethylene glycol in an amount of about 0.2-25% weight/weight; a gelling agent in an amount of about 0.05-5% weight/weight, and an antioxidant in an amount of about 0.025-2.0% weight/weight, all based on the total weight of the topical diclofenac composition.

In some embodiments, the topical diclofenac composition comprises diclofenac sodium in an amount of about 3% weight/weight; the C2 to C4 alkanol in an amount of about 5-60% weight/weight; the polyalcohol in an amount of about 1-30% weight/weight; the monoalkyl ether of diethylene glycol in an amount of about 0.2-25% weight/weight; a gelling agent in an amount of about 0.05-5% weight/weight, and an antioxidant in an amount of about 0.025-2.0% weight/weight, all based on the total weight of the topical diclofenac composition.

In some embodiments, the topical diclofenac composition comprises diclofenac in an amount from 2.7-3.3% weight/weight; ethanol in an amount from 40.5-49.5% weight/weight; propylene glycol in an amount from 18-22% weight/weight; diethylene glycol monoethyl ether in an amount from 4.5-5.5% weight/weight; and myristyl alcohol in an amount from 2.7-3.3% weight/weight.

In some embodiments, the topical diclofenac composition comprises diclofenac sodium in an amount from 2.7-3.3% weight/weight; ethanol in an amount from 40.5-49.5% weight/weight; propylene glycol in an amount from 18-22% weight/weight; diethylene glycol monoethyl ether in an amount from 4.5-5.5% weight/weight; and myristyl alcohol in an amount from 2.7-3.3% weight/weight.

In some embodiments, the topical diclofenac composition comprises diclofenac in an amount from 2.7-3.3% weight/weight; ethanol in an amount from 40.5-49.5% weight/weight; propylene glycol in an amount from 18-22% weight/weight; diethylene glycol monoethyl ether in an amount from 4.5-5.5% weight/weight; myristyl alcohol in an amount from 2.7-3.3% weight/weight; hydroxypropyl cellulose in an amount from 1.25-1.75% weight/weight; and water.

In some embodiments, the topical diclofenac composition comprises diclofenac sodium in an amount from 2.7-3.3% weight/weight; ethanol in an amount from 40.5-49.5% weight/weight; propylene glycol in an amount from 18-22% weight/weight; diethylene glycol monoethyl ether in an amount from 4.5-5.5% weight/weight; myristyl alcohol in an amount from 2.7-3.3% weight/weight; hydroxypropyl cellulose in an amount from 1.25-1.75% weight/weight; and water.

In some embodiments, the topical diclofenac composition comprises diclofenac in an amount of 3.06% weight/weight; ethanol in an amount of 46.0% weight/weight; propylene glycol in an amount of 20% weight/weight; diethylene glycol monoethyl ether in an of 5.0% weight/weight; myristyl alcohol in an amount of 3.0% weight/weight; and hydroxypropyl cellulose in an amount of 1.5% weight/weight; and water.

In some embodiments, the topical diclofenac composition comprises diclofenac sodium in an amount of 3.06% weight/weight; ethanol in an amount of 46.0% weight/weight; propylene glycol in an amount of 20% weight/weight; diethylene glycol monoethyl ether in an of 5.0% weight/weight; myristyl alcohol in an amount of 3.0% weight/weight; and hydroxypropyl cellulose in an amount of 1.5% weight/weight; and water.

The composition may be prepared in any form suitable for topical application to a skin surface, such as in the form of a gel, lotion, cream, spray, aerosol, ointment, emulsion, suspension, liposomal system, lacquer, patch, bandage, or occlusive dressing. In some embodiments, the composition is in the form of a gel, lotion, cream, spray, aerosol, ointment, emulsion, suspension, liposomal system, or lacquer suitable for topical application to a skin surface. In some embodiments, the composition is in the form of a gel suitable for topical application to a skin surface. In specific embodiments, the composition is in the form of a clear transparent gel.

In some embodiments, the composition is in the form of at least one of the following: a clear (transparent) formulation, a water washable formulation, a cool-to-the-touch formulation, a quick-drying formulation, a spreadable formulation and a non-greasy formulation.

### Methods/Uses

Also provided herein are therapeutic methods using a composition as described herein. In specific embodiments, the methods comprise once daily administration of a composition as describe herein to a subject in need thereof, such as by topically applying (administering) to the skin of a subject a therapeutically effective amount of a topical diclofenac composition as described herein. In specific embodiments, the subject is suffering from muscle or joint pain. In specific embodiments, the subject is suffering from osteoarthritis. In specific embodiments, the subject has been diagnosed with osteoarthritis. A composition as described herein may be applied directly to the skin, such as when formulated as a gel, lotion, ointment, emulsion, suspension, cream, liposomal system, lacquer, spray, aerosol, occlusive dressing (some embodiments), or the like, or may be applied indirectly, such as when formulated in a patch, bandage, occlusive dressing (some embodiments), or the like.

In accordance with specific embodiments, provided herein are methods for treating signs and symptoms of osteoarthritis of a joint amenable to topical treatment, comprising topically applying once daily to an affected area of a subject in need thereof a therapeutically effective amount of a topical diclofenac composition as described herein. In some embodiments, the joint is a joint of the knee, foot, ankle, hand, wrist, or elbow. In some embodiments, the joint is a hip, shoulder, or joint of the spine. In specific embodiments, the joint is a knee. In other specific embodiments, the joint is an elbow. In other specific embodiments, the joint is a joint of the hand. In embodiments where the subject has more than one affected joint (such as suffering from pain, including osteoarthritis pain, in more than one joint, such as in both knees, in one knee and one elbow, etc.), the methods described herein may comprise once daily administration as described herein to an affected area of each joint (e.g., to both knees, to the affect knee and elbow, etc.).

The amount of composition administered once daily may be any amount effective to treat joint pain or signs and symptoms of osteoarthritis of a joint. Typical amounts may range from amounts that provide a daily dose of diclofenac per joint of from about 30 mg to about 100 mg, including a daily dose of diclofenac per joint of from about 30 mg to 110 mg, or a daily dose of diclofenac per joint of from about 30 mg to about 110 mg, based on diclofenac sodium. Typically, the amount may depend on the joint being treated, with a smaller amount being therapeutically effective to treat smaller joints (e.g., elbow or joint of the hand, ankle, foot), and larger amounts being therapeutically effective to treat larger joints (e.g., knee, hip, shoulder) or joint of a spine. Additionally or alternatively, the amount may depend on the condition being treated, e.g., the severity of the pain or signs and symptoms of osteoarthritis.

In some embodiments for treating signs and symptoms of osteoarthritis of a knee, the once daily application provides a daily dose of diclofenac per joint (e.g., per knee) of from about 60 mg to about 100 mg, including from about 60 mg to 110 mg, or a daily dose of diclofenac per joint (e.g., per knee) of from about 60 mg to about 110 mg, based on diclofenac sodium, including about 60, about 65 mg, about 70 mg, about 75 mg, about 80 mg, about 85 mg, about 90 mg, about 95 mg, about 100 mg, including 90-110 mg, or about 110 mg, including 110 mg, based on diclofenac sodium. In some embodiments, the once daily application per joint (e.g., per knee) provides a daily dose of diclofenac per joint of about 70 mg, including 70 mg, based on diclofenac sodium. In some embodiments, the once daily application per joint (e.g., per knee) provides a daily dose of diclofenac per joint of about 100 mg, based on diclofenac sodium, such as 90-110 mg. In some embodiments, the once daily application per joint (e.g., per knee) provides a daily dose of diclofenac per joint of about 110 mg, including 110 mg, based on diclofenac sodium. In some embodiments for treating signs and symptoms of osteoarthritis of, e.g., a hip, shoulder, or joint of a spine, the once daily application provides a daily dose of diclofenac per joint (e.g., per hip, shoulder, or joint of a spine) of from about 60 mg to about 100 mg, based on diclofenac sodium, including about 60, about 65 mg, about 70 mg, about 75 mg, about 80 mg, about 85 mg, about 90 mg, about 95 mg, or about 100 mg, including 90-110 mg, or about 110 mg, including 110 mg, based on diclofenac sodium. In some embodiments, the once daily application per joint (e.g., per hip, shoulder, or joint of a spine) provides a daily dose of diclofenac per joint of about 70 mg, including 70 mg, based on diclofenac sodium. In some embodiments, the once daily application per joint (e.g., per hip, shoulder, or joint of a spine) provides a daily dose of diclofenac per joint of about 100 mg, based on diclofenac sodium, such as 90-110 mg. In some embodiments, the once daily application per joint (e.g., per hip, shoulder, or joint of a spine) provides a daily dose of diclofenac per joint of about 110 mg, including 110 mg, based on diclofenac sodium.

In some embodiments for treating signs and symptoms of osteoarthritis of a smaller joint, such as a joint of the hand, wrist, elbow, foot or ankle, the once daily application provides a daily dose of diclofenac per joint (e.g., per hand, wrist, or elbow) of from about 30 mg to about 70 mg, based on diclofenac sodium, including about 30, about 35 mg, about 40 mg, about 45 mg, about 50 mg, about 55 mg, about 60 mg, about 65 mg, or about 70 mg, based on diclofenac sodium. Alternatively, in some embodiments for treating signs and symptoms of osteoarthritis of a smaller joint, such as a joint of the hand, wrist, elbow, foot or ankle, the once daily application per joint provides a daily dose of diclofenac per joint of about 100 mg, based on diclofenac sodium, such as 90-110 mg, or a daily dose of diclofenac per joint of about 110 mg, including 110 mg, based on diclofenac sodium. In some embodiments, the once daily application per joint (e.g., per hand, wrist, elbow, foot or ankle) provides a daily dose of diclofenac per joint of about 35 mg, including 35 mg, based on diclofenac sodium.

The composition used in a once daily method of treatment as described herein may be any composition described herein above or in the examples below, or any composition described in U.S. Pat. No. 9,999,590 or U.S. Pat. No. 10,117,829, the contents of which are hereby incorporated herein by reference. In some embodiments, the composition is in the form of a gel suitable for topical application to a skin surface. In specific embodiments, the composition is in the form of a clear transparent gel.

For treating signs and symptoms of osteoarthritis of a joint, the therapeutically effective amount of a topical diclofenac composition as described herein (e.g., comprising at least 2.7% weight/weight or from 2.7-3.3% weight/weight or about 3.0% weight/weight, diclofenac), that is applied once daily per joint is from about 0.75 to about 2.5 grams, or from about 0.75 to about 3.5 grams.

In some embodiments for treating signs and symptoms of osteoarthritis of a knee, the therapeutically effective amount of the topical diclofenac composition as described herein that is applied once daily per joint (e.g., per knee) is from about 1.5 to about 2.5 grams, or from about 1.5 to about 3.5 grams, of the composition, including about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, or about 3.5 grams of the composition. In some embodiments, the therapeutically effective amount of the topical diclofenac composition applied once daily to a knee is about 2.3 grams of the composition. In some embodiments, the therapeutically effective amount of the topical diclofenac composition applied once daily to a knee is about 3.5 grams of the composition. In some embodiments for treating signs and symptoms of osteoarthritis of, e.g., a hip, shoulder, or joint of a spine, the therapeutically effective amount of the topical diclofenac composition as described herein that is applied once daily per joint (e.g., per hip, shoulder, or joint of a spine) is from about 1.5 to about 2.5 grams, or from about 1.5 to about 3.5 grams, of the composition, including about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, or about 3.5 grams of the composition. In some embodiments, the therapeutically effective amount of the topical diclofenac composition applied once daily to, e.g., a hip, shoulder, or joint of a spine, is about 2.3 grams of the composition. In some embodiments, the therapeutically effective amount of the topical diclofenac composition applied once daily to, e.g., a hip, shoulder, or joint of a spine, is about 3.5 grams of the composition.

In some embodiments for treating signs and symptoms of osteoarthritis of a smaller joint, such as a joint of the hand, wrist, elbow, foot or ankle, the therapeutically effective amount of the topical diclofenac composition as described herein that is applied once to a joint (e.g., to a hand, wrist, or elbow) is from about 0.75 to about 1.5 grams of the composition, including about 0.75, about 0.8, about 0.9, about 1.0, about 1.1, about 1.2, about 1.3, about 1.4, or about 1.5 of the composition. In some embodiments, the therapeutically effective amount of the topical diclofenac composition applied once daily to e.g., a hand, wrist, or elbow, is about 1.15 grams of the composition. Alternatively, in some embodiments for treating signs and symptoms of osteoarthritis of a smaller joint, such as a joint of the hand, wrist, elbow, foot or ankle, the therapeutically effective amount of the topical diclofenac composition as described herein that is applied once daily per joint is from about 1.5 to about 2.5 grams of the composition, including about 1.5, about 1.6, about 1.7, about 1.8, about 1.9, about 2.0, about 2.1, about 2.2, about 2.3, about 2.4, or about 2.5 grams of the composition. In some embodiments, the therapeutically effective amount of the topical diclofenac composition applied once daily to a smaller joint is about 2.3 grams of the composition.

In some embodiments, the composition is applied from a metered dose dispenser. For example, a composition in the form of a gel, lotion, cream, spray, aerosol, ointment, emulsion, suspension, liposomal system, or lacquer may be applied from a metered dose dispenser. In some embodiments, the metered dose dispenser comprises a metered dose pump. In any embodiments wherein the composition is applied from a metered dose dispenser that comprises a metered dose pump, the therapeutically effective amount (e.g., daily dose per joint) of the topical diclofenac composition may be provided by one or two or more actuations of the metered dose pump, such as one, two, three, or more actuations of the metered dose pump. In some such embodiments for treating a knee, the therapeutically effective amount of the topical diclofenac composition is provided by two actuations of the metered dose pump. In some such embodiments for treating a knee, the therapeutically effective amount of the topical diclofenac composition is provided by three actuations of the metered dose pump. In some such embodiments for treating, e.g., a hip, shoulder, or joint of a spine, the therapeutically effective amount of the topical diclofenac composition is provided by two actuations of the metered dose pump. In some such embodiments for treating, e.g., a hip, shoulder, or joint of a spine, the therapeutically effective amount of the topical diclofenac composition is provided by three actuations of the metered dose pump. In some such embodiments, such as for treating a smaller joint, the therapeutically effective amount of the topical diclofenac composition is provided by one actuation of the metered dose pump. In some embodiments, the composition is in the form of a gel, and is applied from a metered dose dispenser, such as metered dose dispenser that comprise a metered dose pump, wherein a therapeutically effective amount (e.g., daily dose per joint) of the topical diclofenac gel composition may be provided by one or two or more actuations of the metered dose pump, such as one, two, three, or more actuations of the metered dose pump, such as one or two or three actuations of the metered dose pump, depending on the joint being treated and/or condition thereof.

In some embodiments, a once daily method as disclosed herein is effective for treating joint pain, including osteoarthritis pain of a joint. In some embodiments, a once daily method as described herein is effective for relief of pain, such as pain of osteoarthritis, as determined by one or more of the tools for assessing pain described below.

A primary tool for assessing osteoarthritis pain is the Western Ontario and McMaster Osteoarthritis Index (WOMAC). See, e.g., Bellamy et al., “Validation study of WOMAC: a health status instrument for measuring clinically important patient relevant outcomes to antirheumatic drug therapy in patients with osteoarthritis of the hip or knee,” J Rheumatol. 1988; 15:1833-1840. A WOMAC assessment may be made before and after treatment to assess treatment of pain, as illustrated in Example 2 below. The WOMAC questionnaire is widely used in clinical trials of osteoarthritis treatments and has been extensively validated. The maximum possible total score, indicating the worst possible osteoarthritis symptoms, is 240 points.

TABLE 1

| WOMAC scores | Range (min-max) |
|---|---|
| Total score | 0-240 |
| Pain sub-score | 0-50 |
| Pain weight-bearing sub-score | 0-30 |
| Pain non-weight-bearing sub-score | 0-20 |
| Stiffness sub-score | 0-20 |
| Function sub-score | 0-170 |

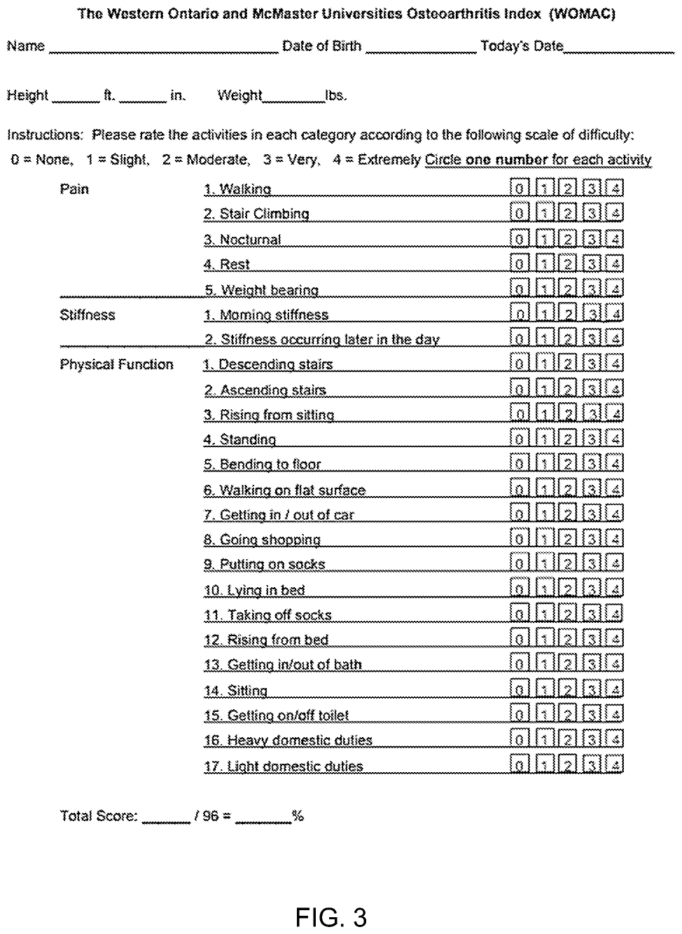

The 24-question WOMAC questionnaire addresses the degree of pain experienced with different activities or positions (5 questions=pain sub-score), the degree and timing of joint stiffness (2 questions=stiffness sub-score), and the degree of difficulty experienced in performing daily activities (i.e., physical function) (17 questions=function sub-score). See, e.g., FIG. 3.

All WOMAC scores discussed herein (including the sub-scores) are normalized to a 0 to 100-point scale (normalized score=(score/max possible value)*100). The minimal clinically important difference (MCID) of the WOMAC function-sub score has been reported to be approximately 10 out of 100; thus, a clinically important change in WOMAC sub-score, such as the WOMAC pain sub-score, as assessed herein may be considered to a normalized sub-score of 10 out of 100 (e.g., raw pain sub-score of 5 out of 50). In some embodiments, the subject before treatment has a WOMAC pain sub-score of 40 or greater (≥40) on a normalized scale of 0 to 100. In some embodiments, the subject before treatment has a WOMAC pain sub-score of 90 or less (≤90) on a normalized scale of 0 to 100. In some embodiments, the subject before treatment has a WOMAC pain sub-score of ≥40 and ≤90 on a normalized scale of 0 to 100.

In some embodiments, a once daily method as disclosed herein is effective for relief of pain, such as pain of osteoarthritis, as determined by any one or more of (i) a decrease in the subject's WOMAC pain sub-score after treatment as compared to before treatment; (ii) a decrease in the subject's WOMAC physical function sub-score after treatment as compared to before treatment; (iii) a decrease in the subject's WOMAC stiffness sub-score after treatment as compared to before treatment; (iv) a decrease in the subject's WOMAC pain weight-bearing sub-score after treatment as compared to before treatment; (v) a decrease in the subject's WOMAC pain non-weight-bearing sub-score after treatment as compared to before treatment; and (vi) a decrease in the subject's WOMAC total score after treatment as compared to before treatment. Examples of such results are illustrated in Example 2 below.

Other tools for assessing pain (and relief thereof) include the EuroQol-5 Domain Questionnaire (EQ-5D questionnaire), which is a standardized generic measure of health-related quality of life (QoL). The questionnaire asks about overall health today in five dimensions: mobility, self-care, usual activities, pain/discomfort and anxiety/depression, each measured on five levels (no problems, slight problems, moderate problems, severe problems and extreme problems) and a Visual Analog Scale (VAS) of the subject's self-rated health. In some embodiments, a once daily method as disclosed herein is effective for relief of pain, as determined by EQ-5D assessment before and after treatment, such as an EQ-5D VAS assessment before and after treatment.

Another tool for assessing pain (and relief thereof) is the Work Productivity and Activity Impairment (WPAI) questionnaire. The WPAI is a patient-reported outcome (PRO) instrument used to evaluate productivity/absenteeism due to a specific disease. The instrument consists of the below 6 questions; one question relating to employment status, and five questions relating to productivity and hours missed due to the disease in question and other reasons. WPAI outcomes are expressed as impairment percentages, with higher numbers indicating greater impairment and less productivity, i.e., worse outcomes.

1. Currently employed (yes/no)

2. Hours missed due to health problems

3. Hours missed other reasons

4. Hours actually worked

5. Degree health affected productivity while working (11-point scale from 0 to 10)

6. Degree health affected regular activities (11-point scale from 0 to 10) WPAI-derived endpoints may include one or more of Work Time Missed (Q2/(Q2+Q4)); Impairment While Working (Q5/10); Overall Work Impairment (Q2/(Q2+Q4)+[(1−(Q2/(Q2+Q4)))×(Q5/10)]); and Activity Impairment (Q6/10). In some embodiments, a once daily method as disclosed herein is effective for relief of pain, as determined by WPAI assessment of any one or more WPAI-derived endpoints before and after treatment, such as Activity Impairment and/or Overall Work Impairment.

In some embodiments, an assessment “after treatment” by any one or more of the tools outlined above is conducted after treatment once daily for one week, four weeks, eight weeks, twelve weeks, or longer. Additionally, or alternatively, the assessment “after treatment” is conducted after treatment once daily for one, two, three, four, five, six, eight, ten, twelve, sixteen, twenty-four, thirty-six, forty-eight, and/or ninety-six weeks, etc. For example, assessment after treatment, such as WOMAC pain sub-score assessment and/or any other assessment described herein, may be conducted after treatment once daily for a period of time selected from 1-4 weeks, 4-8 weeks, 8-12, weeks, or longer.

In some embodiments, the effectiveness of the method in relieving pain, such as pain of osteoarthritis, is at least as effective as twice daily administration of the same amount of the same topical diclofenac composition, such as may be determined by any one or more of the assessments outlined above, such as WOMAC pain sub-score, WOMAC total score, or any one or more other WOMAC sub-scores, before and after treatment, such as by a greater decrease in the subject's WOMAC pain sub-score after once daily treatment as compared to any decrease reported after twice daily treatment, and/or by EQ-5D and/or WPAI assessment. To assess such relative efficacy, the once daily and twice daily treatments may be carried out in the same subject or in the same pool of subjects (e.g., in a cross-over design), or in comparable subjects or pools of subjects (e.g., in a randomized multi-arm design). In some embodiments, the assessment “after treatment” is conducted after treatment once daily for four weeks. Additionally or alternatively, the assessment “after treatment” is conducted after treatment once daily for one, two, three, four, five, six, eight, ten, twelve, sixteen, twenty-four, thirty-six, forty-eight, and/or ninety-six weeks, etc.

The once daily administration may be at any time of day. In some embodiments, the once daily administration is in the morning, such as before commencing daily activities. In some embodiments, the once daily administration is in the evening, such as before going to sleep. For a method of treatment comprising repeated once daily administrations, the once daily administration may at about the same time of day each day, or may be at a different time of day each day. In some embodiments of a method of treatment comprising repeated once daily administrations, the once daily administration is at about the same time of day.

Treatment by the once daily methods disclosed herein may be carried out for as long as the subject is in need thereof, such as for 1, 2, 3, 4, 5, 6, or 7 days, 1, 2, 3, 4, 5, or 6 weeks, 1, 2, 3, 4, 5, or 6 months, or longer, including 9 months, 12 months, 18 months, 24 months, 36 months, 48 months, or longer, etc. When a once daily method as described herein is used to treat a chronic condition, such as osteoarthritis, the treatment period may continue after the subject has experienced clinically important improvement, such as clinically important pain relief. For example, the treatment may be ongoing to provide ongoing relief of chronic pain.

In specific embodiments, a topical diclofenac composition as disclosed herein is used to treat subjects with osteoarthritis of a joint, such as a knee, hip, shoulder or joint of the spine, in a once daily protocol that provides a dose of 90-110 mg diclofenac per joint per day, based on diclofenac sodium. For example, a composition as disclosed herein containing 2.7-3.3% (w/w) diclofenac sodium is applied once daily in an amount to provide a dose of 90-110 mg diclofenac per joint per day. Such a composition may be provided in a metered dose dispenser, such as a metered dose dispenser that dispenses about 1 gram of composition per actuation, such as 1.15 gram of composition per actuation, and administered once daily by applying a number of pump actuations of the composition onto the skin surface of the affected joint once a day that will provide a total treatment application of 90-110 mg diclofenac sodium per joint per day (such as three pump actuations). Further application instructions optionally may include to spread and rub the gel in, in a gentle manner, onto the anterior, medial and lateral (not posterior) surfaces of the affected knee(s), not cover the application site with clothes for at least 10 minutes after application, and avoid showering or bathing for at least 4 hours after application. In a very specific illustrative example, the topical diclofenac gel composition of Formula I of Example 1, Table 2 below (or a comparable formulation having a similar amount of diclofenac sodium) is dispensed once daily by three actuations from a metered dose dispenser that dispenses about 1 gram of composition per actuation, such as 1.15 gram per actuation, to apply a once daily dose of diclofenac sodium of about 105 mg (3.45 g×3.06%=105.6 mg). These once daily protocols will be effective to treat OA assessed by one or more or all of the endpoints discussed herein.

### Kits

Also provided are kits comprising one or more containers containing a topical diclofenac composition as described herein. In some embodiments, the kit comprises a container that is a metered dose dispenser optionally comprising a metered dose pump (such as described above) containing one or more daily doses per joint of a topical diclofenac composition as described herein. In other embodiments, the kit comprises one or more containers, each containing a single daily dose (e.g., a daily dose for a single joint) of a topical diclofenac composition as described herein. In other embodiments, the kit comprises one or more containers, each containing multiple daily doses (e.g., multiple daily doses for a single joint or a single daily dose for multiple joints or multiple daily doses for multiple joints) of a topical diclofenac composition as described herein.

The composition provided in a kit as described herein may be any composition as described herein above or in the examples below, or any composition described in U.S. Pat. No. 9,999,590 or U.S. Pat. No. 10,117,829, the contents of which are hereby incorporated herein by reference.

In some embodiments, the kit further comprises instructions for topically applying once daily to an affected area of a subject in need thereof a therapeutically effective amount of the composition, such as any of the doses of diclofenac/amounts of composition discussed above. In some embodiments, the kit further comprises instructions for once daily treatment of signs and symptoms of osteoarthritis of a joint amenable to topical treatment, comprising topically applying once daily to an affected area of a subject in need thereof a therapeutically effective amount of the composition. When the container is a metered dose dispenser that comprise a metered dose pump, the instructions may advise to dispense a therapeutically effective amount (e.g., daily dose per joint) of the topical diclofenac gel composition by one or two or three or more actuations of the metered dose pump, such as by one or two or three actuations of the metered dose pump, depending on the joint being treated and/or condition thereof. The instructions may advise to perform the once daily treatment at about the same time each day. Application instructions may advise to spread and rub the gel in, in a gentle manner, not cover the application site with clothes for at least 10 minutes after application, and avoid showering or bathing for at least 4 hours after application.

### EXAMPLES

The following specific examples are included as illustrative of the compositions and methods described herein. These examples are in no way intended to limit the scope of the disclosure. Other aspects of the disclosure will be apparent to those skilled in the art to which the disclosure pertains.

### Example 1

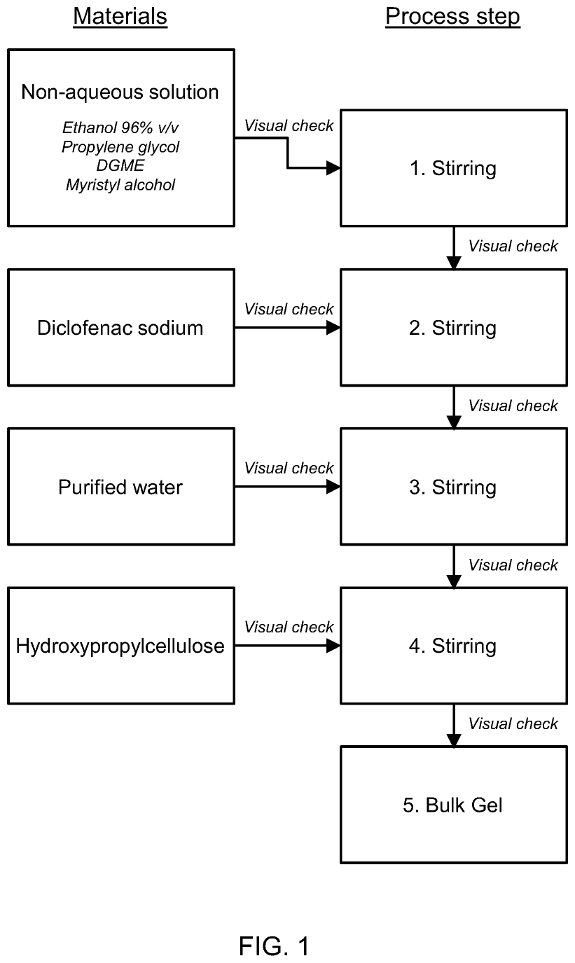

The diclofenac compositions described below are prepared as shown schematically in FIG. 1. For bench scale (10 grams to 1000 grams batch sizes), preparation can be carried out in amber glass bottles, with mixing and homogenization by magnetic stirring or marine propellers. For larger batch scale (e.g., 6 kg and beyond, e.g., 6-10 kg, 60-100 kg, or 600-1000 kg), preparation can be carried out in a controlled environment under continuous vacuum and nitrogen blanketing with temperature control, into a stainless steel planetary mixer.

Exemplary diclofenac compositions are shown in the below table.

TABLE 2

| | Formulations (% weight/weight) | | | | | | | | |
|---|---|---|---|---|---|---|---|---|---|
| Components | A | B | C | D | E | F | G | H | I |
| Diclofenac sodium | 3.0 | 3.0 | 3.15 | 3.0 | 3.0 | 3.15 | 3.0 | 3.0 | 3.06 |
| Absolute Ethanol | 45.0 | 45.0 | 45.0 | 45.0 | 45.0 | 45.0 | 40.5 | 49.5 | 46.0* |
| Propylene glycol | 20.0 | 20.0 | 20.0 | 20.0 | 20.0 | 20.0 | 18.0 | 22.0 | 20 |
| Diethylene glycol | 5.0 | 5.0 | 5.0 | 5.0 | 5.0 | 5.0 | 4.5 | 5.5 | 5 |
| monoethyl ether | | | | | | | | | |
| Myristyl alcohol | 2.0 | 3.0 | 3.0 | 3.0 | 3.0 | 2.0 | 2.7 | 3.3 | 3 |
| (USP/NF) | | | | | | | | | |
| Hydroxypropylcellulose | 1.50 | 1.50 | 1.5 | 1.5 | 1.5 | 1.5 | 1.5 | 1.5 | 1.5 |
| Sodium metabisulfite | — | — | — | 0.1 | 0.05 | — | — | — | — |
| Propyl gallate | — | — | — | — | 0.05 | — | — | — | — |
| Purified water | qs | qs | qs | qs | qs | qs | qs | qs | qs |
| | 100 | 100 | 100 | 100 | 100 | 100 | 100 | 100 | 100 |

*Provided as 47.9% ethanol 96% v/v.

While these examples comprise diclofenac sodium, similar compositions can be formulated with 2-(2,6-dichloranilino) phenylacetic acid (“diclofenac acid”) or any other pharmaceutically acceptable salt thereof. Such compositions could be formulated with the same amount of 2-(2,6-dichloranilino) phenylacetic acid or pharmaceutically acceptable salt thereof as listed above for diclofenac sodium (e.g., 3.0, 3.15, or 3.06% weight/weight), or with an amount that provides an equivalent amount of the diclofenac moiety, taking into account the molecular weight of the form used. For example, 3.0% weight/weight diclofenac sodium is equivalent to 2.79% weight/weight diclofenac acid; 3.15% weight/weight diclofenac sodium is equivalent to 2.93% weight/weight diclofenac acid; and 3.06% weight/weight diclofenac sodium is equivalent to 2.85% weight/weight diclofenac acid.

Voltaren® Gel 1% includes the following ingredients, with 1.00% weight/weight diclofenac sodium.

TABLE 3
Voltaren ® Gel 1% Components

Diclofenac sodium
Carbomer homopolymer Type C
Cocoyl caprylocaprate (Cetiol LC)
Isopropyl alcohol
Fragrance
Mineral oil (liquid paraffin)
Polyoxyl 20 cetostearyl ether
Propylene glycol
Strong ammonium solution
Water purified

### Example 2

### Study Design

This study was a multi-center, double-blind, randomized, placebo-controlled trial of a topical diclofenac composition as described herein for the treatment of knee osteoarthritis (“OA”) symptoms. The trial also included a single-blind component, consisting of Voltaren® Gel 1%. The aim of the trial was to evaluate the efficacy and safety and tolerability of once or twice daily application of a topical diclofenac composition as described herein during a 28-day period in subjects with radiographic and symptomatic knee OA in either one or both knees. (If both knees met the inclusion criteria, both knees could be treated, but one knee was selected as the “target” knee throughout the study.) The primary objective was to evaluate the change in pain intensity, as assessed by the WOMAC pain sub-score of the target knee. A total of 444 subjects were randomized in a ratio of 3:3:3:2 with 121 subjects in each of the three double-blinded treatment groups and 81 subjects in the single-blinded treatment group.

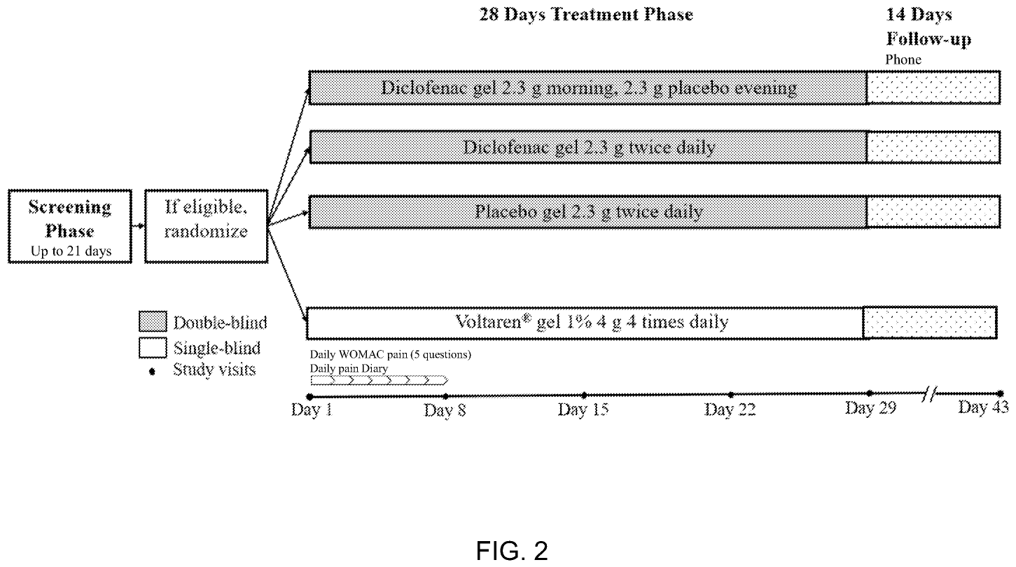

The trial consisted of 3 phases: a screening period of up to 21 days, a treatment period of 28 days, and a follow-up period of 14 days, as illustrated in FIG. 2.

The main inclusion criteria were femorotibial osteoarthritis of the knee, as determined by clinical and radiographic criteria (e.g., Kellgren-Lawrence radiographic severity of 1-3), and WOMAC pain sub-score (5 questions) of ≥40 and ≤90 out of 100 at the time of screening (Intent-To-Treat, or “ITT” group). The subjects were randomized to apply a topical diclofenac gel composition as described herein once daily (QD) using 2.3 g of an “active” topical diclofenac gel composition as described herein in the morning and a placebo in the evening, or twice daily (BID) using 2.3 g of the “active” topical diclofenac gel composition as described herein in the morning and in the evening, or a placebo twice daily, all per OA knee, in a double-blind fashion for a period of 28 days, or to apply 4 g of Voltaren® Gel 1% four times daily (QID) in a single-blind fashion for exploratory comparison.

The primary endpoint was change from baseline (before treatment vs. after treatment) in subject-reported pain on the WOMAC pain sub-score in the target knee. Secondary endpoints included WOMAC function and stiffness sub-scores, WOMAC pain weight-bearing sub-score, WOMAC pain non-weight-bearing sub-score, WOMAC total pain score, EuroQol-5 Domain Questionnaire (EQSD) VAS scores, and Work Productivity and Activity Impairment (WPAI) scores. Safety endpoints included nature, incidence and severity of adverse effects (AEs); changes in laboratory safety parameters, vital signs, 12-lead ECG parameters, and weight; and nature, incidence, and severity of skin reactions on the application site.

As noted above, the selection criteria included a WOMAC pain sub-score of ≥40 at the time of screening. However, screening could take place up to 3 weeks prior to the baseline visit. It was observed that some subjects, although meeting the pain criteria at screening, presented with a WOMAC pain sub-score less than 20 at the baseline visit. Because that may limit the magnitude of their potential improvement during the trial, a post-hoc subgroup analysis was conducted for subjects who had a WOMAC normalized pain sub-score of ≥40 out of 100 at baseline (week 0).

### WOMAC Questionnaire

WOMAC assessment before and after treatment by the self-administered 24-question WOMAC questionnaire Version 3.1 (see FIG. 3) was used to determine the change from baseline in the target knee at week 4 (after 4 weeks treatment). Each question was scored on an 11-point numerical rating scale (from 0 to 10), where higher numbers indicate greater pain, stiffness or difficulty in performing daily activities. As noted above, the maximum possible WOMAC total score, indicating the worst possible OA symptoms, is 240 points. As noted above, for convenience and ease of interpretation, all scores (including sub-scores) were normalized to a 0 to 100-point scale for data analysis. The minimal clinically important difference (MCID) of the WOMAC function sub-score has been reported to be approximately 10 out of 100. Therefore, as noted above, a clinically important change in the WOMAC pain sub-score (5 questions) as assessed herein was defined as 5 out of 50 points (10 out of 100 on the normalized scale).

As noted above, change in the WOMAC pain sub-score (questions 1 to 5) was used as the primary endpoint, while secondary endpoints included change from baseline at week 4 in WOMAC total score, WOMAC physical function and stiffness sub-scores, WOMAC pain weight-bearing sub-score and WOMAC pain non-weight-bearing sub-score.

### EuroQol-5 Domain Questionnaire

As another secondary endpoint, change from baseline in quality of life (QoL) as assessed by the EQ-5D questionnaire were evaluated at week 4 (after four weeks of treatment). The Visual Analog Scale (VAS) of the subject's self-rated health was scored from 0-100 where 0 corresponds to “the worst health you can imagine” and 100 corresponds to “the best health you can imagine.” The EQ-5D was assessed at baseline (visit 2), week 3 (visit 5) and week 4 (visit 6) (after four weeks of treatment).

### Work Productivity Activity Impairment Scale

As another secondary endpoint, changes from baseline in work productivity and activity were assessed by the WPAI questionnaire at week 4 (after four weeks of treatment). WPAI outcomes are expressed as impairment percentages, with higher numbers indicating greater impairment and less productivity, i.e., worse outcomes. WPAI was assessed at baseline (visit 2) and weeks 1 (visit 3), 2 (visit 4) and 4 (visit 6) (4 weeks after treatment).

### Investigational Products and Dosing

As noted above, the study used a topical diclofenac composition as described herein, namely, the topical diclofenac gel composition of Formula I of Example 1, Table 2, above (referred to below as “Diclofenac Gel”). The composition was a completely homogeneous, transparent and non-staining hydroalcoholic gel, containing 3.06% diclofenac sodium. The placebo was a visually identical gel composition having the same components except the diclofenac sodium. Both were provided in 87-gram metered dose dispensers that dispensed 1.15 gram gel per actuation. Commercially available Voltaren® Gel 1% was sourced from the U.S. market in 100 g tubes.

Subjects allocated to the double-blinded treatment arms (assessing treatment once or twice daily with a topical diclofenac gel composition as described herein versus placebo) were instructed to apply two pump actuations of gel (2×1.15 grams=2.3 grams) onto the skin surface of the affected knee(s) at approximately the same time each morning and evening. (The time between morning and evening applications was to be approximately 10-12 hours, e.g., 7 a.m. and 5 p.m.) On visit days, the gel was not to be applied in the morning prior to a visit. Further application instructions included to spread and rub the gel in, in a gentle manner, onto the anterior, medial and lateral (not posterior) surfaces of the affected knee(s), not cover the application site with clothes for at least 10 minutes after application, and avoid showering or bathing for at least 4 hours after application. Thus, each treatment application applied 70 mg diclofenac sodium per knee, such that subjects in the once daily treatment group applied 70 mg diclofenac sodium daily (2.3 g×3.06%=70 mg), while subjects in the twice daily treatment group applied 140 mg diclofenac sodium daily.

For the subjects allocated to treatment with Voltaren® Gel 1%, the approved product labelling was followed, such that each subject was instructed to apply 4 grams of gel using the dosing card enclosed in the treatment kit, four times during the day directly onto the skin surface of the affected knee(s). The time points at which the gel was applied were to be distributed throughout the day and cover the morning (e.g., 7 a.m.), noon (e.g., 12 a.m.), evening (e.g., 5 p.m.), and before bedtime (e.g., 10 p.m.), separating each application by approximately 5 hours. On visit days, the gel was not to be applied in the morning prior to a visit. Thus, subjects treated with Voltaren® Gel 1%, applied 16 mg diclofenac sodium per affected knee daily.

A total of 444 subjects were randomized. The subject disposition and main baseline characteristics are shown in Table 4.

TABLE 4
Subject Disposition and Baseline Characteristics

| | | Diclofenac | Diclofenac | | Voltaren ® | |
|---|---|---|---|---|---|---|
| | | Gel BID | Gel QD | Placebo | 1% | Total |
| | | (N = 120) | (N = 120) | (N = 120) | (N = 78) | (N = 438) |
| Completed¹ (n %) | | 114 (94.2) | 109 (90.1) | 108 (89.3) | 70 (86.4) | 401 (90.3) |
| Discontinued¹ (n %) | | 7 (5.8) | 12 (9.9) | 13 (10.7) | 11 (13.6) | 43 (9.7) |
| Mean age, years (SD) | | 63.3 (10.5) | 64.4 (9.3) | 63.2 (9.3) | 65.5 (8.9) | 64.0 (9.6) |
| Female, % | | 68.3 | 68.3 | 64.2 | 65.4 | 66.7 |
| Race, % | White | 95.0 | 98.3 | 95.0 | 94.9 | 95.9 |
| | Black or | 5.0 | 0.8 | 3.3 | 3.8 | 3.2 |
| | African | | | | | |
| | American | | | | | |
| | Asian or | 0 | 0.8 | 1.7 | 1.3 | 0.9 |
| | Pacific | | | | | |
| | Islander | | | | | |
| Mean BMI (SD) | | 30.6 (5.0) | 31.1 (5.1) | 31.2 (5.3) | 31.0 (5.4) | 31.0 (5.2) |
| Mean WOMAC² | | 56.4 (12.18) | 54.8 (10.52) | 54.4 (9.60) | 54.8 (10.38) | 55.2 (10.74) |
| pain sub-score at | | | | | | |
| screening, target | | | | | | |
| knee, SD | | | | | | |
| Unilateral knee OA, % | | 68.3 | 63.3 | 66.7 | 74.4 | 67.6 |
| Bilateral knee OA, % | | 31.7 | 36.7 | 33.3 | 25.6 | 32.4 |
| Kellgren- | KL 1 | 19.2 | 14.2 | 20.0 | 10.3 | 16.4 |
| Lawrence | KL 2 | 35.8 | 29.2 | 35.8 | 30.8 | 33.1 |
| (KL) score | KL 3 | 45.0 | 56.7 | 43.3 | 59.0 | 50.2 |
| of target | | | | | | |
| knee at | | | | | | |
| screening, % | | | | | | |

¹Numbers are for the Intent-To-Treat (ITT) group, i.e., the 444 subjects randomized and treated. Other data in this table are for the modified Intent-To-Treat (mITT) group which does not include 6 randomized subjects (1 from Diclofenac Gel QD group, 1 from Diclofenac Gel BD group, 1 from placebo group and 3 from Voltaren ® group) whose results were not analyzed due to missing post-baseline WOMAC data.
²Reported on normalized scale of 0-100

### Primary Endpoints

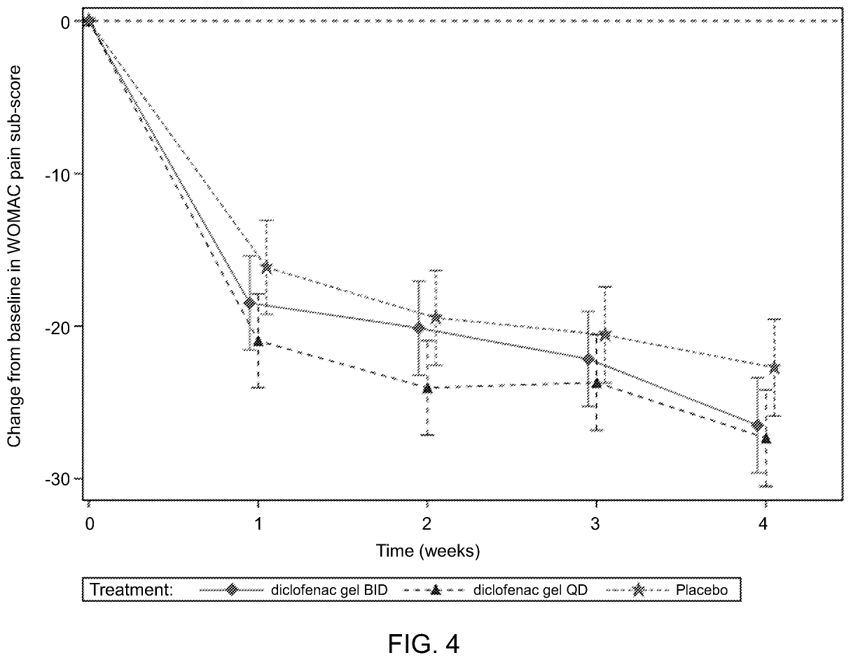

For all mITT subjects, across all groups, the mean normalized WOMAC pain sub-score at baseline was approximately 52-53, with scores ranging from a minimum value of 8 in the Diclofenac Gel BID group to a maximum value of 86 in the Diclofenac Gel QD group. The observed mean change in normalized WOMAC pain sub-score from baseline to week 4 ranged from approximately −23 in the placebo group to −27 in the Diclofenac Gel BID group and −28 in the Diclofenac Gel QD group, where reduced scores represent an improvement in pain. Results are shown in FIG. 4, Table 5, and Table 6, which summarize WOMAC pain sub-score change from baseline to week 4. These results show the most significant treatment effect for the Diclofenac Gel QD (once daily) treatment group versus placebo (statistically significant).

TABLE 5
Change in Baseline in WOMAC Pain Sub-Score

| | Diclofenac | Diclofenac | | Voltaren ® |
|---|---|---|---|---|
| | Gel BID | Gel QD | Placebo | 1% |
| | (N = 121) | (N = 121) | (N = 121) | (N = 81) |
| | n (%) | n (%) | n (%) | n (%) |
| Baseline | | | | |
| Score | | | | |
| Mean (SD) | 53.0 (13.7) | 52.6 (12.7) | 52.1 (12.3) | 53.5 (13.8) |
| Median | 50.0 | 50.0 | 52.0 | 52.0 |
| Week 1 | | | | |
| Change | | | | |
| Mean (SD) | −19.1 (17.1) | −20.9 (17.0) | −15.6 (15.5) | −19.7 (17.5) |
| Median | −17.0 | −20.0 | −15.0 | −18.0 |
| Week 2 | | | | |
| Change | | | | |
| Mean (SD) | −21.1 (18.3) | −24.7 (17.5) | −19.1 (16.7) | −20.1 (17.9) |
| Median | −21.0 | −26.0 | −18.0 | −18.0 |
| Week 3 | | | | |
| Change | | | | |
| Mean (SD) | −22.8 (18.5) | −24.8 (19.1) | −20.9 (18.5) | −23.8 (17.0) |
| Median | −24.0 | −26.0 | −20.0 | −24.0 |
| Week 4 | | | | |
| Change | | | | |
| Mean (SD) | −26.9 (19.2) | −28.0 (19.6) | −22.9 (18.3) | −26.7 (17.2) |
| Median | −26.0 | −28.0 | −22.0 | −24.0 |

TABLE 6
WOMAC Pain Sub-Score Treatment Comparison

| | Treatment | | |
|---|---|---|---|
| Treatment Comparison | Difference | [95% CI] | p-value |
| Diclofenac Gel BID vs. Placebo | −3.76 | [−8.21, 0.68] | 0.0969 |
| Diclofenac Gel QD vs. Placebo | −4.61 | [−9.09, −0.12] | 0.0440 |
| Diclofenac Gel BID vs. | 0.84 | [−3.60, 5.28] | 0.7098 |
| Diclofenac Gel QD | | | |
| Diclofenac Gel BID vs. | −0.74 | [−5.70, 4.23] | 0.7709 |
| Voltaren ® 1% | | | |
| Diclofenac Gel QD vs. | −1.52 | [−6.52, 3.49] | 0.5525 |
| Voltaren ® 1% | | | |
| Voltaren ® Gel 1% vs. Placebo | −3.07 | [−8.08, 1.94] | 0.2292 |

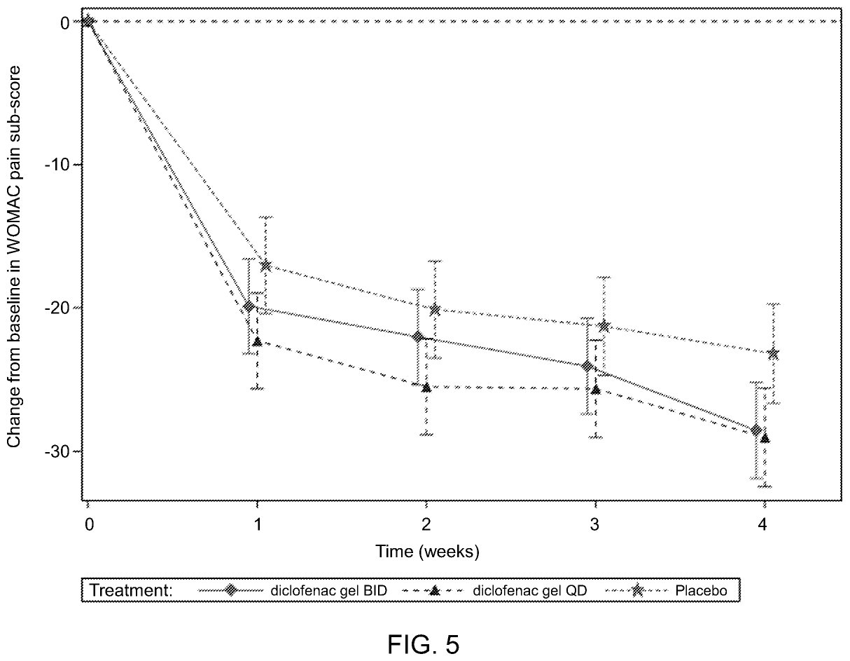

For the sub-population of subjects with WOMAC score ≥40 out of 100 at screening and baseline (the post-hoc analysis subgroup), the mean normalized WOMAC pain sub-score was approximately 55-56 at baseline in all treatment groups. The WOMAC pain sub-score improved in all groups from baseline to week 4 with a mean change between approximately −24 and −30. The mean change in normalized WOMAC pain sub-score from baseline to week 4 was statistically significantly greater for both Diclofenac Gel groups (QD and BID) versus placebo, as shown in FIG. 5 and Table 7.

TABLE 7
WOMAC Pain Sub-Score Treatment Comparison for
Post-Hoc Analysis Subgroup with WOMAC Score ≥
40 out of 100 at Screening and Baseline

| | Treatment | | |
|---|---|---|---|
| Treatment Comparison | Difference | [95% CI] | p-value |
| Diclofenac Gel BID vs. Placebo | −5.35 | [−10.16, −0.54] | 0.0292 |
| Diclofenac Gel QD vs. Placebo | −5.84 | [−10.71, −0.97] | 0.0189 |
| Diclofenac Gel BID vs. | 0.49 | [−4.30, 5.27] | 0.8410 |
| Diclofenac Gel QD | | | |
| Diclofenac Gel BID vs. | −0.92 | [−6.32, 4.47] | 0.7366 |
| Voltaren ® 1% | | | |
| Diclofenac Gel QD vs. | −1.38 | [−6.84, 4.08] | 0.6193 |
| Voltaren ® 1% | | | |
| Voltaren ® Gel 1% vs. Placebo | −4.47 | [−9.95, 1.01] | 0.1096 |

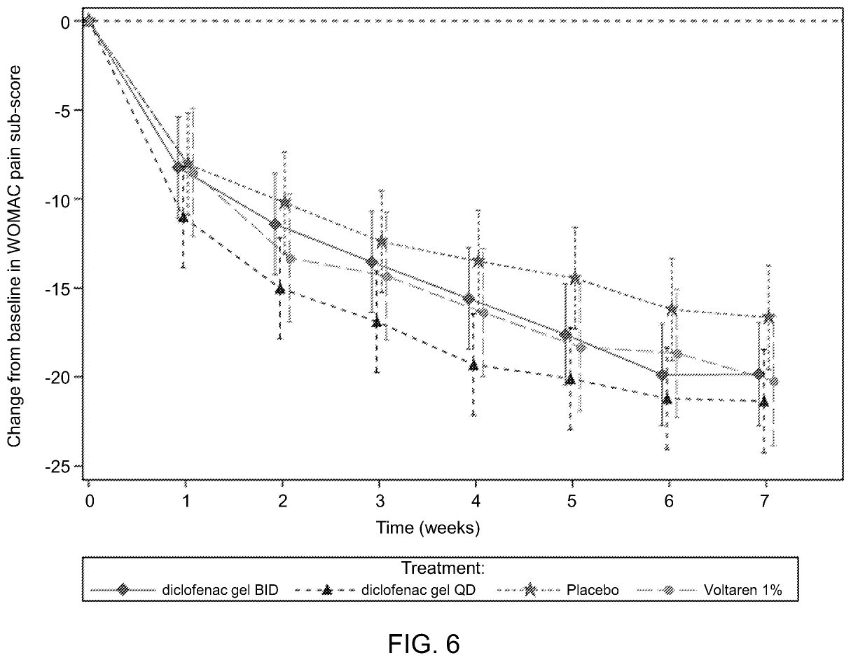

FIG. 6 and Table 8 summarize the WOMAC pain sub-score change from baseline during and after the first week of treatment for all mITT subjects. These results show a statistically significant difference between the Diclofenac Gel QD (once daily) group and placebo group after the first week of treatment.

TABLE 8
WOMAC Pain Sub-Score Treatment Comparison
After First Week of Treatment

| | Treatment | | |
|---|---|---|---|
| Treatment Comparison | Difference | [95% CI] | p-value |
| Diclofenac Gel BID vs. Placebo | −3.19 | [−7.31, 0.92] | 0.1279 |
| Diclofenac Gel QD vs. Placebo | −4.70 | [−8.82, −0.58] | 0.0255 |
| Diclofenac Gel BID vs. | 1.51 | [−2.60, 5.61] | 0.4718 |
| Diclofenac Gel QD | | | |
| Diclofenac Gel BID vs. | 0.37 | [−4.28, 5.03] | 0.8751 |
| Voltaren ® 1% | | | |
| Diclofenac Gel QD vs. | −1.13 | [−5.80, 3.54] | 0.6340 |
| Voltaren ® 1% | | | |
| Voltaren ® Gel 1% vs. Placebo | −3.57 | [−8.24, 1.11] | 0.1344 |

### Secondary Endpoints

Table 9 summarizes the WOMAC total score change from baseline at 4 weeks (after 4 weeks treatment) for all mITT subjects. Across all treatment groups, the normalized mean WOMAC total score at baseline was approximately 50-52 and the observed mean changes from baseline to week 4 were in the range of −22 to −24, indicating improvements. These results show that change in baseline in both the Diclofenac Gel QD (once daily) and BID (twice daily) groups were greater than the change in baseline for the placebo group.

TABLE 9
WOMAC Total Score Treatment Comparison

| | Treatment | | |
|---|---|---|---|
| Treatment Comparison | Difference | [95% CI] | p-value |
| Diclofenac Gel BID vs. Placebo | −3.58 | [−7.58; 0.42] | 0.0789 |
| Diclofenac Gel QD vs. Placebo | −2.75 | [−6.78; 1.28] | 0.1802 |
| Diclofenac Gel BID vs. | −0.83 | [−4.83; 3.17] | 0.6837 |
| Diclofenac Gel QD | | | |
| Diclofenac Gel BID vs. | −0.88 | [−5.34, 3.58] | 0.6990 |
| Voltaren ® 1% | | | |
| Diclofenac Gel QD vs. | −0.02 | [−4.51, 4.48] | 0.9938 |
| Voltaren ® 1% | | | |
| Voltaren ® 1% vs. Placebo | −2.72 | [−7.22, 1.77] | 0.2345 |

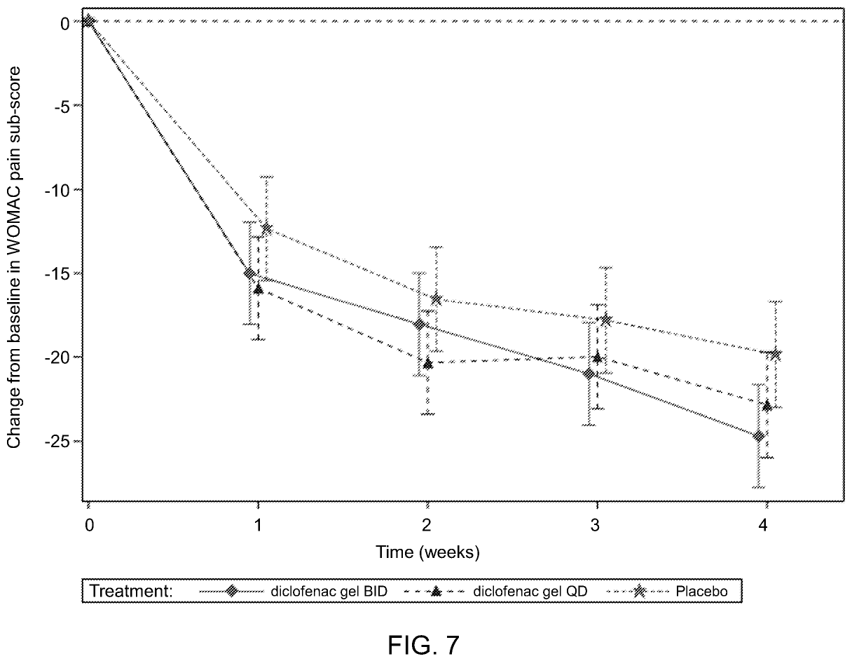

FIG. 7 and Table 10 summarize the WOMAC function sub-score change from baseline at 4 weeks (after 4 weeks treatment). Across all treatment groups, the normalized mean WOMAC function sub-score at baseline was approximately 49-51 and observed mean changes from baseline to week 4 were in the range of −21 to −24, indicating improvements. These results show that change in baseline in both the Diclofenac Gel QD (once daily) and BID (twice daily) groups were greater than the change in baseline for the placebo group.

TABLE 10
WOMAC Function Sub-Score Treatment Comparison

| | Treatment | | |
|---|---|---|---|
| Treatment Comparison | Difference | [95% CI] | p-value |
| Diclofenac Gel BID vs. Placebo | −3.50 | [−7.57; 0.57] | 0.0921 |
| Diclofenac Gel QD vs. Placebo | −2.36 | [−6.46; 1.74] | 0.2593 |
| Diclofenac Gel BID vs. | −1.14 | [−5.21; 2.93] | 0.5834 |
| Diclofenac Gel QD | | | |
| Diclofenac Gel BID vs. | −0.93 | [−5.47, 3.61] | 0.6867 |
| Voltaren 1% | | | |
| Diclofenac Gel QD vs. | 0.24 | [−4.33, 4.82] | 0.9164 |
| Voltaren 1% | | | |
| Voltaren ® 1% vs. Placebo | −2.58 | [−7.16, 1.99] | 0.2676 |

Table 11 summarizes the WOMAC stiffness sub-score change from baseline to week 4 (after 4 weeks treatment). Across all treatment groups, the normalized mean WOMAC stiffness sub-score at baseline was approximately 50-53 and observed mean changes from baseline to week 4 were in the range of −21 to −24. These results show that change in baseline in both Diclofenac Gel groups (QD and BID) were greater than for the placebo group.

TABLE 11
WOMAC Stiffness Sub-Score Treatment Comparison

| | Treatment | | |
|---|---|---|---|
| Treatment Comparison | Difference | [95% CI] | p-value |
| Diclofenac Gel BID vs. Placebo | −2.52 | [−7.16; 2.11] | 0.2854 |
| Diclofenac Gel QD vs. Placebo | −2.70 | [−7.38; 1.97] | 0.2567 |
| Diclofenac Gel BID vs. | 0.18 | [−4.45; 4.81] | 0.9394 |
| Diclofenac Gel QD | | | |
| Diclofenac Gel BID vs. | −0.67 | [−5.89, 4.55] | 0.8011 |
| Voltaren ® 1% | | | |
| Diclofenac Gel QD vs. | −0.80 | [−6.07, 4.46] | 0.7642 |
| Voltaren ® 1% | | | |
| Voltaren ® 1% vs. Placebo | −1.87 | [−7.14, 3.39] | 0.4848 |

Table 12 summarizes the WOMAC pain weight-bearing sub-score change from baseline to week 4 (after 4 weeks treatment). Across all treatment groups, the normalized mean WOMAC pain weight-bearing sub-score at baseline was approximately 56-57 (slightly higher than the overall pain sub-score). Observed mean changes from baseline to week 4 ranged from approximately −23 in the placebo group to approximately −28 in both Diclofenac Gel groups. These results show that change in baseline in both Diclofenac Gel groups (QD and BID) groups were greater than the change in baseline for the placebo group, with the difference for Diclofenac Gel QD (once daily) being statistically significant.

TABLE 12
WOMAC Pain Weight-Bearing Sub-Score Treatment Comparison

| | Treatment | | |
|---|---|---|---|
| Treatment Comparison | Difference | [95% CI] | p-value |
| Diclofenac Gel BID vs. Placebo | −4.38 | [−9.14; 0.38] | 0.0710 |
| Diclofenac Gel QD vs. Placebo | −5.03 | [−9.83; −0.24] | 0.0396 |
| Diclofenac Gel BID vs. | 0.65 | [−4.10; 5.40] | 0.7877 |
| Diclofenac Gel QD | | | |
| Diclofenac Gel BID vs. | −0.57 | [−5.88, 4.75] | 0.8341 |
| Voltaren ® 1% | | | |
| Diclofenac Gel QD vs. | −1.15 | [−6.51, 4.22] | 0.6748 |
| Voltaren ® 1% | | | |
| Voltaren 1% ® vs. Placebo | −3.86 | [−9.23, 1.51] | 0.1583 |

Table 13 summarizes the WOMAC pain non-weight-bearing sub-score change from baseline to week 4 (after 4 weeks treatment). Across all treatment groups, the normalized mean WOMAC pain non-weight-bearing sub-score at baseline was approximately 46 (slightly lower than the overall pain sub-score). Observed mean changes from baseline to week 4 ranged from approximately −23 in the placebo group to −28 in the Diclofenac Gel QD (once daily) group. These results show that change in baseline in both Diclofenac Gel groups (QD and BID) were greater than the change in baseline for the placebo group.

TABLE 13
WOMAC Pain Non-Weight-Bearing
Sub-Score Treatment Comparison

| | Treatment | | |
|---|---|---|---|
| Treatment Comparison | Difference | [95% CI] | p-value |
| Diclofenac Gel BID vs. Placebo | −2.72 | [−7.32; 1.88] | 0.2460 |
| Diclofenac Gel QD vs. Placebo | −3.96 | [−8.60; 0.68] | 0.0944 |
| Diclofenac Gel BID vs. | 1.24 | [−3.35; 5.83] | 0.5961 |
| Diclofenac Gel QD | | | |
| Diclofenac Gel BID vs. | −1.00 | [−6.15, 4.14] | 0.7014 |
| Voltaren ® 1% | | | |
| Diclofenac Gel QD vs. | −2.20 | [−7.39, 2.99] | 0.4054 |
| Voltaren ® 1% | | | |
| Voltaren ® 1% vs. Placebo | −1.74 | [−6.93, 3.44] | 0.5094 |

Table 14 summarizes the ED-5D overall quality of life change from baseline to week 4 (after 4 weeks treatment). Across all treatment groups, the baseline mean VAS score was between approximately 64 and 67 out of 100, where higher scores represent better health. The VAS score was improved from baseline to week 4 with a mean increase in observed score of approximately 11-12 in all treatment groups.

TABLE 14
ED-5D Overall Quality of Life Treatment Comparison

| | Treatment | | |
|---|---|---|---|
| Treatment Comparison | Difference | [95% CI] | p-value |
| Diclofenac Gel BID vs. Placebo | 4.70 | [0.55, 8.85] | 0.0264 |
| Diclofenac Gel QD vs. Placebo | 3.43 | [−0.74, 7.60] | 0.1072 |
| Diclofenac Gel BID vs. | 1.27 | [−2.86, 5.41] | 0.5459 |
| Diclofenac Gel QD | | | |
| Diclofenac Gel BID vs. | 1.25 | [−3.43, 5.93] | 0.6014 |
| Voltaren ® 1% | | | |
| Diclofenac Gel QD vs. | −0.06 | [−4.77, 4.65] | 0.9812 |
| Voltaren ® 1% | | | |
| Voltaren ® 1% vs. Placebo | 3.44 | [−1.27, 8.14] | 0.1518 |

Table 15 summarize the WPAI % Activity Impairment from baseline to week 4 (after 4 weeks treatment). At baseline, all groups had an average impairment in their regular activities of almost 50% due to health problems. At week 4, the mean % Activity Impairment was noticeably reduced by approximately 14-20% points in all treatment groups. These results show a statistically significant difference between the Diclofenac Gel QD (once daily) group and placebo.

TABLE 15
WPAI % Activity Impairment Treatment Comparison

| | Treatment | | |
|---|---|---|---|
| Treatment Comparison | Difference | [95% CI] | p-value |
| Diclofenac Gel BID vs. Placebo | −4.72 | [−10.00, 0.55] | 0.0790 |
| Diclofenac Gel QD vs. Placebo | −7.39 | [−12.69, −2.08] | 0.0064 |
| Diclofenac Gel BID vs. | 2.66 | [−2.59, 7.92] | 0.3202 |
| Diclofenac Gel QD | | | |
| Diclofenac Gel BID vs. | −3.23 | [−9.03, 2.57] | 0.2750 |
| Voltaren ® 1% | | | |
| Diclofenac Gel QD vs. | −5.89 | [−11.72, −0.05] | 0.0479 |
| Voltaren ® 1% | | | |
| Voltaren ® 1% vs. Placebo | −1.51 | [−7.35, 4.33] | 0.6120 |

Table 16 summarizes the WPAI % Overall Work Impairment from baseline to week 4 (after 4 weeks treatment). At baseline, all groups had some degree of overall work impairment due to health problems. At week 4, the % Overall Work Impairment was reduced in all treatment groups. These results show a statistically significant difference between the Diclofenac Gel QD (once daily) group and placebo.

TABLE 16
WPAI % Overall Work Impairment Treatment Comparison

| | Treatment | | |
|---|---|---|---|
| Treatment Comparison | Difference | [95% CI] | p-value |
| Diclofenac Gel BID vs. Placebo | −5.20 | [−15.17, 4.77] | 0.3054 |
| Diclofenac Gel QD vs. Placebo | −10.44 | [−20.84, −0.04] | 0.0492 |
| Diclofenac Gel BID vs. | 5.24 | [−4.20, 14.68] | 0.2752 |
| Diclofenac Gel QD | | | |
| Diclofenac Gel BID vs. | −4.75 | [−15.68, 6.19] | 0.3933 |
| Voltaren ® 1% | | | |
| Diclofenac Gel QD vs. | −9.88 | [−21.17, 1.41] | 0.0860 |
| Voltaren ® 1% | | | |
| Voltaren ® 1% vs. Placebo | −0.48 | [−12.18, 11.21] | 0.9352 |

### Diclofenac Exposure

Table 17 summarizes the diclofenac plasma concentration data at week 2 and week 4. The geometric mean diclofenac concentration was lowest in the Diclofenac Gel QD (once daily) group, intermediate in the Voltaren Gel 1% group, and highest in the Diclofenac Gel BID (twice daily) group. In the three active treatment groups, the geometric mean diclofenac plasma concentrations were slightly lower at week 4 than week 2. At both week 2 and week 4, the diclofenac concentration was statistically significantly higher in the Diclofenac Gel BID group compared to the Diclofenac Gel QD group. At week 2, but not at week 4, the plasma concentration in the Diclofenac Gel QD group was lower than in the Voltaren Gel 1% group. These results show that once daily treatment with a diclofenac composition as described herein results in less systemic exposure to diclofenac than other treatment regimens.

TABLE 17
Diclofenac Plasma Concentration

| | | Diclofenac | Diclofenac | | Voltaren ® |
|---|---|---|---|---|---|
| Diclofenac plasma | | Gel BID | Gel QD | Placebo | 1% |
| concentration (ng/mL) | | (n = 120) | (n = 121) | (n = 120) | (n = 78) |
| Week 2 | n | 112 | 113 | 114 | 115 |
| | Mean (SD) | 13.8 (12.7) | 7.8 (6.2)* | 0.7 (2.8) | 10.6 (7.4) |
| | Median | 10.8 | 6.0 | 0.1 | 10.2 |
| | Geometric mean | 9.2 (154.2) | 5.5 (115.2)* | 0.1 (144.1) | 7.8 (122.8) |
| | (CV(%)) | | | | |
| | n | 110 | 110 | 104 | 72 |
| Week 4 | Mean (SD) | 13.4 (10.6) | 7.5 (5.8)** | 2.2 (18.0) | 10.8 (8.8) |
| | Median | 11.9 | 6.5 | 0.1 | 9.0 |
| | Geometric mean | 8.8 (171.7) | 5.3 (120.0)** | 0.1 (177.1) | 6.8 (184.3) |
| | (CV(%)) | | | | |

CV, coefficient of variable
For reference, Cmax of Voltaren ® tablet 3 × 50 mg daily = 2270 ng/mL
*Statistical significance vs. Diclofenac Gel BID (p < 0.001) and vs. Voltaren ® 1% (p < 0.05);
**Statistical significance vs. Diclofenac Gel BID (p < 0.001)

Assessment of other safety and tolerability parameters were favorable, as the frequency of adverse events leading to discontinuation of treatment was similarly low (ranging between 2.8% to 6.6%) across all treatments. The most common treatment-emergent adverse events were application site dryness, with a frequency of 10.7% for Diclofenac Gel QD, 14.9% for Diclofenac Gel BID, 13.2% for vehicle, and 6.2% for Voltaren® Gel 1%, and application site erythema, with a frequency of 12.4% for Diclofenac Gel QD, 9.1% for Diclofenac Gel BID, 33.1% for vehicle, and 4.9% for Voltaren® Gel 1%, suggesting a slight irritant effect of the vehicle, which may be reduced by diclofenac. No serious adverse events were reported during the trial.

### Conclusion

The results show that once daily treatment with a topical diclofenac composition as described herein was better than placebo in every endpoint discussed above. Moreover, once daily treatment with a topical diclofenac composition as described herein was statistically significantly better in treating pain than placebo as assessed by the following: WOMAC pain sub-score change from baseline to week 4 (FIG. 4, Table 5); WOMAC pain sub-score change from baseline to week 4 for the sub-population with WOMAC pain sub-score ≥40 at screening and baseline (post-hoc subgroup) (FIG. 5, Table 7); WOMAC pain sub-score from baseline during and after the first week of treatment (FIG. 6 and Table 8, respectively); WOMAC pain weight-bearing sub-score change from baseline to week 4 (Table 12); WPAI % Activity Impairment from baseline to week 4 (Table 15); and WPAI % Overall Work Impairment from baseline to week 4 (Table 16).

The results also showed no statistically significant difference between treatment once daily versus twice daily with the same topical diclofenac composition as described herein, indicating that once daily treatment with a topical diclofenac composition as described herein is at least as effective as twice daily treatment with the same topical diclofenac composition.

Viewed collectively, these results show that once daily treatment with a topical diclofenac composition as described herein provides effective treatment of osteoarthritis knee pain, with less systemic exposure to diclofenac than other treatment regimens, with a good tolerability and safety profile. The results show that the once daily treatment methods described herein are safe and effective, and surprisingly indicate they are at least as effective as twice daily treatment with the same topical diclofenac composition (e.g., when assessed by WOMAC pain sub-score), even though once daily treatment involves administration of half the daily dose of diclofenac as compared to twice daily treatment, and results in much lower systemic exposure to diclofenac, as illustrated in Table 17.

### Example 3

A topical diclofenac composition as disclosed in Formula I of Example 1, Table 2, above, is used to treat subjects with osteoarthritis of the knee in one or both knees, in a protocol that provides a once daily dose of 90-110 mg diclofenac per knee, based on diclofenac sodium. In particular, the topical diclofenac gel composition of Formula I of Example 1, Table 2 is dispensed once daily by three pump actuations from a metered dose dispenser as used in Example 2 (which dispenses 1.15 g composition per actuation) to apply a once daily dose of diclofenac sodium of about 105 mg (3.45 g×3.06%=105.6 mg). Further application instructions may include to spread and rub the gel in, in a gentle manner, onto the anterior, medial and lateral (not posterior) surfaces of the affected knee(s), not cover the application site with clothes for at least 10 minutes after application, and avoid showering or bathing for at least 4 hours after application. This once daily protocol will be effective to treat OA assessed by one or more or all of the endpoints discussed above.

What is claimed is:

1. A method for treating signs and symptoms of osteoarthritis of a joint amenable to topical treatment, comprising topically applying once daily to an affected area of a subject in need thereof a therapeutically effective amount of a topical diclofenac composition, wherein the composition comprises:
(i) at least 2.7% weight/weight of diclofenac;
(ii) a C2 to C4 alkanol;
(iii) a polyalcohol;
(iv) a monoalkyl ether of diethylene glycol; and
(v) from about 3% to about 5% weight/weight of a fatty alcohol,
all based on the total weight of the topical diclofenac composition,
wherein the once daily application provides a daily dose of diclofenac per joint of from about 30 mg to about 100 mg, based on diclofenac sodium.

2. The method of claim 1, wherein the method is effective for relief of pain of osteoarthritis.

3. The method of any one the preceding claims, wherein the method is effective for relief of pain of osteoarthritis as determined by Western Ontario and McMaster Osteoarthritis Index (WOMAC) assessment before and after treatment.

4. The method of claim 3, wherein the method is effective for relief of pain of osteoarthritis as determined by one or more of:
a decrease in the subject's WOMAC pain sub-score after treatment as compared to before treatment;
a decrease in one or more of the subject's WOMAC physical function sub-score and WOMAC stiffness sub-score after treatment as compared to before treatment;
a decrease in the subject's WOMAC weight-bearing pain sub-score after treatment as compared to before treatment;
a decrease in the subject's WOMAC non-weight-bearing pain sub-score after treatment as compared to before treatment; and
a decrease in the subject's WOMAC total score after treatment as compared to before treatment.

5. The method of any one of the preceding claims, wherein before treatment the subject has a WOMAC pain sub-score of 40 or greater when normalized to a scale of 0 to 100.

6. The method of any one of the preceding claims, wherein before treatment the subject has a WOMAC pain sub-score of 40 or greater and 90 or less when normalized to a scale of 0 to 100.

7. The method of any one of the preceding claims, wherein the method is at least as effective for relief of pain of osteoarthritis as compared to twice daily administration of the same amount of the topical diclofenac composition, as determined by WOMAC pain sub-score assessment before and after treatment.

8. The method of any one of claims 3-7, wherein WOMAC assessment after treatment is conducted after treatment once daily for a period of time selected from 1-4 weeks, 4-8 weeks, 8-12, weeks, or longer.

9. The method of any one of the preceding claims, wherein the topical diclofenac composition further comprises at least one of a gelling agent, an anti-oxidant, a solvent and any combinations thereof.

10. The method of any one of the preceding claims, wherein the topical diclofenac composition comprises:
the diclofenac in an amount of about 3% weight/weight;
the C2 to C4 alkanol in an amount of about 5-60% weight/weight;
the polyalcohol in an amount of about 1-30% weight/weight;
the monoalkyl ether of diethylene glycol in an amount of about 0.2-25% weight/weight;
a gelling agent in an amount of about 0.05-5% weight/weight, and
an antioxidant in an amount of about 0.025-2.0% weight/weight,
all based on the total weight of the topical diclofenac composition.

11. The method of any one of the preceding claims, wherein, in the topical diclofenac composition:
the C2 to C4 alkanol is selected from ethanol, isopropanol, n-propanol, butan-1-ol, and butan-2-ol;
the polyalcohol is selected from glycol, propylene glycol, butylene glycol, and hexylene glycol;
the monoalkyl ether of diethylene glycol is selected from diethylene glycol monoethyl ether, diethylene glycol monomethyl ether, and combinations thereof; and
the fatty alcohol is selected from myristyl alcohol, lauryl alcohol, oleyl alcohol, cetyl alcohol, and stearyl alcohol.

12. The method of any one of the preceding claims, wherein the topical diclofenac composition comprises diclofenac sodium.

13. The method of any one of the preceding claims, wherein the topical diclofenac composition comprises:
diclofenac sodium in an amount from 2.7-3.3% weight/weight;
ethanol in an amount from 40.5-49.5% weight/weight;
propylene glycol in an amount from 18-22% weight/weight;
diethylene glycol monoethyl ether in an amount from 4.5-5.5% weight/weight; and
myristyl alcohol in an amount from 2.7-3.3% weight/weight.

14. The method of any one of the preceding claims, wherein the topical diclofenac composition comprises:
diclofenac sodium in an amount of about 3.0% weight/weight;
ethanol in an amount from 40.5-49.5% weight/weight;
propylene glycol in an amount from 18-22% weight/weight;
diethylene glycol monoethyl ether in an amount from 4.5-5.5% weight/weight; and
myristyl alcohol in an amount from 2.7-3.3% weight/weight.

15. The method of any one of the preceding claims, wherein the topical diclofenac composition comprises:
diclofenac sodium in an amount from 2.7-3.3% weight/weight;
ethanol in an amount from 40.5-49.5% weight/weight;
propylene glycol in an amount from 18-22% weight/weight;
diethylene glycol monoethyl ether in an amount from 4.5-5.5% weight/weight;
myristyl alcohol in an amount from 2.7-3.3% weight/weight;
hydroxypropyl cellulose in an amount from 1.25-1.75% weight/weight; and
water.

16. The method of any one of the preceding claims, wherein the topical diclofenac composition comprises:
diclofenac sodium in an amount of 3.06% weight/weight;
ethanol in an amount of 46.0% weight/weight;
propylene glycol in an amount of 20% weight/weight;
diethylene glycol monoethyl ether in an of 5.0% weight/weight;
myristyl alcohol in an amount of 3.0% weight/weight;
hydroxypropyl cellulose in an amount of 1.5% weight/weight; and
water.

17. The method of any one of the preceding claims, wherein the therapeutically effective amount of the topical diclofenac composition applied once daily per joint is from about 0.75 to about 2.50 grams.

18. The method of any one of the preceding claims, wherein the joint is selected from a knee, elbow, or joint of a foot, ankle, hand, wrist, hip, shoulder, or spine.

19. The method of any one of the preceding claims, wherein the joint is a knee, and the once daily application provides a daily dose of diclofenac per knee of from about 60 mg to about 100 mg, based on diclofenac sodium.

20. The method of claim 19, wherein the once daily application provides a daily dose of diclofenac of about 70 mg per knee, based on diclofenac sodium.

21. The method of claim 19 or 20, wherein the therapeutically effective amount of the topical diclofenac composition applied once daily per knee is from about 1.5 to about 2.5 grams.

22. The method of any one of claims 1-18, wherein the once daily application provides a daily dose of diclofenac per joint of from about 30 mg to 110 mg, based on diclofenac sodium.

23. The method of any one of claim 1-18 or 22, wherein the therapeutically effective amount of the topical diclofenac composition applied once daily per joint is from about 0.75 to about 3.5 grams.

24. The method of any one of claims 1-18, wherein the once daily application provides a daily dose of diclofenac per joint of 90-110 mg, based on diclofenac sodium, optionally wherein the joint is a knee.

25. The method of claim 24, wherein the therapeutically effective amount of the topical diclofenac composition applied once daily per joint is about 3.5 grams, optionally wherein the joint is a knee.

26. The method of claim 18, wherein the joint is selected from a hip, shoulder, or joint of the spine, and the once daily application provides a daily dose of diclofenac per joint of from about 60 mg to about 100 mg, based on diclofenac sodium.

27. The method of claim 26, wherein the once daily application provides a daily dose of diclofenac of about 70 mg per joint, based on diclofenac sodium.

28. The method of claim 26, wherein the once daily application provides a daily dose of diclofenac per joint of 90-110 mg, based on diclofenac sodium.

29. The method of claim 28, wherein the therapeutically effective amount of the topical diclofenac composition applied once daily per joint is about 3.5 grams.

30. The method of any one of claims 1-18, wherein the joint is an elbow, or joint of a foot, ankle, hand, or wrist, and the once daily application provides a daily dose of diclofenac per joint of from about 30 mg to about 70 mg, based on diclofenac sodium.

31. The method of claim 30, wherein the once daily application provides a daily dose of diclofenac of about 35 mg per joint, based on diclofenac sodium.

32. The method of claim 30 or 31, wherein the therapeutically effective amount of the topical diclofenac composition applied once daily per joint is from about 0.75 to about 1.50 grams.

33. The method of any one of the preceding claims, wherein the topical diclofenac composition is in a form selected from a gel, lotion, cream, spray, aerosol, ointment, emulsion, suspension, liposomal system, lacquer, patch, bandage, and occlusive dressing.

34. The method of any one of the preceding claims, wherein the topical diclofenac composition is in a form selected from a gel, lotion, cream, spray, aerosol, ointment, emulsion, suspension, liposomal system, and lacquer.

35. The method of any one of the preceding claims, wherein the topical diclofenac composition is in the form of a gel.

36. The method of claim 34 or 35, wherein the topical diclofenac composition is applied from a metered dose dispenser.

37. The method of claim 36, wherein the metered dose dispenser comprises a metered dose pump.

38. The method of claim 37, wherein the therapeutically effective amount of the topical diclofenac composition is provided by one or two or three or more actuations of the metered dose pump.

39. A kit for once daily treatment of signs and symptoms of osteoarthritis of a joint amenable to topical treatment, comprising at least one container containing a topical diclofenac composition, wherein the composition comprises:
diclofenac sodium in an amount of 3.06% weight/weight;
ethanol in an amount of 46.0% weight/weight;
propylene glycol in an amount of 20% weight/weight;
diethylene glycol monoethyl ether in an of 5.0% weight/weight;
myristyl alcohol in an amount of 3.0% weight/weight;
hydroxypropyl cellulose in an amount of 1.5% weight/weight; and
water.

40. The kit of claim 39, further comprising instructions for topically applying once daily to an affected area of a subject in need thereof a therapeutically effective amount of the composition that provides a daily dose of the diclofenac sodium of from about 30 mg to about 100 mg per joint.

41. The kit of claim 39 or 40, wherein the container is a metered dose dispenser.

42. The kit of claim 41, wherein the metered dose dispenser comprises a metered dose pump.

43. The kit of claim 42, wherein the therapeutically effective amount of the topical diclofenac composition is provided by one or two or three or more actuations of the metered dose pump.

44. A topical diclofenac composition for use in treating signs and symptoms of osteoarthritis of a joint amenable to topical treatment by once daily application to an affected area of a subject in need thereof, wherein the composition comprises:
(i) at least 2.7% weight/weight of diclofenac;
(ii) a C2 to C4 alkanol;
(iii) a polyalcohol;
(iv) a monoalkyl ether of diethylene glycol; and
(v) from about 3% to about 5% weight/weight of a fatty alcohol,
all based on the total weight of the topical diclofenac composition,
wherein the once daily application provides a daily dose of diclofenac per joint of from about 30 mg to about 100 mg, based on diclofenac sodium.

45. The composition according to claim 44, wherein the joint is selected from a knee, elbow, or joint of a foot, ankle, hand, wrist, hip, shoulder, or spine.

46. The composition according to claim 44 or 45, wherein the joint is a knee, and the once daily application provides a daily dose of diclofenac per knee of from about 60 mg to about 100 mg, based on diclofenac sodium.

47. The composition according to any one of claims 44-46, wherein the composition is provided in and applied from a metered dose dispenser, optionally wherein the wherein the metered dose dispenser comprises a metered dose pump, optionally wherein the therapeutically effective amount of the topical diclofenac composition is provided by one or two or three or more actuations of the metered dose pump.
